(19) United States
(12) Patent Application Publication
(10) Pub. No.: US 2018/0303535 A1
(43) Pub. Date: Oct. 25, 2018

(54) CRYOABLATION CATHETER HAVING AN ELLIPTICAL-SHAPED TREATMENT SECTION

(71) Applicant: ADAGIO MEDICAL, Inc., Laguna Hills, CA (US)

(72) Inventors: Xiaoyu Yu, San Diego, CA (US); Steven Kovalcheck, San Diego, CA (US); Thomas Chien, Laguna Hills, CA (US); Alexei Babkin, Dana Point, CA (US); Nicolei King, Laguna Hills, CA (US)

(21) Appl. No.: 15/578,649

(22) PCT Filed: May 23, 2016
(86) PCT No.: PCT/US16/33833

§ 371 Date: Nov. 30, 2017

Related U.S. Application Data
(60) Provisional application No. 62/170,243, filed on Jun. 3, 2015.

(51) Int. Cl. A61B 18/02

(52) U.S. Cl. CPC A61B 18/02; A61B 2018/00577; A61B 2018/0268

(57) ABSTRACT

A cryoablation catheter for creating at least one lesion in tissue, the catheter having an elongate shaft with an intermediate section and a distal tip movable relative to the intermediate section. The catheter also includes at least one elongate control member extending along the intermediate section and secured to the distal tip where the elongate control member is movable relative to the intermediate section for causing movement of the distal tip relative to the intermediate section and at least one energy delivery member extending along the intermediate section to the distal tip where the at least one energy delivery member includes a linear first configuration and an elliptical second configuration. Manipulation of the control member adjusts the shape of the at least one energy delivery member.

### CROSS-REFERENCE TO RELATED APPLICATIONS

The present application is a US 371 National Phase filing of International PCT Patent Application No. PCT/US2016/033833 filed May 23, 2016, which claims the benefit of U.S. Provisional Application No. 62/170,243, filed Jun. 3, 2015, the entire contents of which are incorporated herein by reference in their entirety for all purposes.

### BACKGROUND OF THE INVENTION

### 1. Field of the Invention

This invention relates to cryosurgery and more particularly to cryoablation catheters comprising a fluid operating near its critical point.

### 2. Description of the Related Art

Cryosurgery is a promising approach for treating various medical conditions, none of which are less important than the treatment of an abnormal heart beat.

Atrial flutter and atrial fibrillation are heart conditions in which the left or right atrium of the heart beat improperly. Atrial flutter is a condition when the atria beat very quickly, but still evenly. Atrial fibrillation is a condition when the atria beat very quickly, but unevenly.

These conditions are often caused by aberrant electrical behavior of some portion of the atrial wall. Certain parts of the atria, or nearby structures such as the pulmonary veins, can misfire in their production or conduction of the electrical signals that control contraction of the heart, creating abnormal electrical signals that prompt the atria to contract between normal contractions caused by the normal cascade of electrical impulses. This can be caused by spots of ischemic tissue, referred to as ectopic foci, or by electrically active fibers in the pulmonary veins, for example.

The Cox Maze procedure, developed by Dr. James Cox in the 1980's, is a method for eliminating atrial fibrillation. In the Cox Maze procedure, the atrial wall is cut with a scalpel in particular patterns which isolate the foci of arrhythmia from the rest of the atrial wall, and then sewn back together. Upon healing, the resultant scar tissue serves to interrupt ectopic re-entry pathways and other aberrant electrical conduction and prevent arrhythmia and fibrillation. There are several variations of the Cox maze procedure, each involving variations in the number and placement of lesions created.

The original Cox maze procedure was an open chest procedure requiring surgically opening the atrium after opening the chest. The procedure itself has a high success rate, though due to the open chest/open heart nature of the procedure, and the requirement to stop the heart and establish a coronary bypass, it is reserved for severe cases of atrial fibrillation.

The Cox maze procedure has been performed using ablation catheters in both transthoracic epicardial approaches and transvascular endocardial approaches. In transthoracic epicardial approaches, catheters or small probes are used to create linear lesions in the heart wall along lines corresponding to the maze of the Cox maze procedure. In the transvascular endocardial approaches, a catheter is navigated through the vasculature of the patient to the atrium, pressed against the inner wall of the atrium, and energized to create lesions corresponding to the maze of the Cox maze procedure.

In either approach, various ablation catheters have been proposed for creation of the lesion, including flexible cryoprobes or cryocatheters, bipolar RF catheters, monopolar RF catheters (using ground patches on the patient's skin), microwave catheters, laser catheters, and ultrasound catheters. U.S. Pat. No. 6,190,382 to Ormsby and U.S. Pat. No. 6,941,953 to Feld, for example, describe RF ablation catheters for ablating heart tissue. These approaches are attractive because they are minimally invasive and can be performed on a beating heart. However, these approaches have a low success rate. The low success rate may be due to incomplete lesion formation. A fully transmural lesion is required to ensure that the electrical impulse causing atrial fibrillation are completely isolated from the remainder of the atrium, and this is difficult to achieve with beating heart procedures.

A major challenge to the effective epicardial application of ablative energy sources to cardiac tissue without the use of the heart-lung machine (“off-pump”) is that during normal heart function the atria are filled with blood at 37° C. that is moving through the atria at roughly 5 liters per minute. If cryothermia energy is applied epicardially, this atrial blood flow acts as a “cooling sink,” warming the heart wall and making it difficult to lower the endocardial surface of the atrial wall to a lethal temperature (roughly −30° C.). Thus, lesion transmurality is extremely difficult to attain.

Similarly, if heat-based energy sources such as RF, microwave, laser, or HIFU are applied to the epicardial surface without using the heart-lung machine to empty the atria, the blood flowing through the atrium acts as a heat sink, cooling the heart wall making it difficult to raise the endocardial surface of the atrial wall to a lethal temperature (roughly 55° C.).

Another shortcoming with certain cryosurgical apparatus arises from evaporation. The process of evaporation of a liquefied gas results in enormous expansion as the liquid converts to a gas; the volume expansion is on the order of a factor of 200. In a small-diameter system, this degree of expansion consistently results in a phenomenon known in the art as “vapor lock.” The phenomenon is exemplified by the flow of a cryogen in a thin-diameter tube, such as is commonly provided in a cryoprobe. A relatively massive volume of expanding gas that forms ahead of it impedes the flow of the liquid cryogen.

Traditional techniques that have been used to avoid vapor lock have included restrictions on the diameter of the tube, requiring that it be sufficiently large to accommodate the evaporative effects that lead to vapor lock. Other complex cryoprobe and tubing configurations have been used to “vent” N₂ gas as it formed along transport tubing. These designs also contributed to limiting the cost efficacy and probe diameter.

Another challenge for the surgeon is to place the probe along the correct tissue contour. Due to the nature of the procedure and the anatomical locations where the lesions must be placed, the cryoprobe must be sufficiently flexible and adjustable.

Malleable and flexible cryoprobes are described in U.S. Pat. Nos. 6,161,543 and 8,177,780, both to Cox et al. The described probe has a malleable shaft. In embodiments, a malleable metal rod is coextruded with a polymer to form the shaft. The malleable rod permits the user to plastically deform the shaft into a desired shape so that a tip can reach the tissue to be ablated.

U.S. Pat. No. 5,108,390, issued to Potocky et al, discloses a highly flexible cryoprobe that can be passed through a blood vessel and into the heart without external guidance other than the blood vessel itself.

A challenge with some of the above apparatuses, however, is making continuous contact along the anatomical surface such that a continuous lesion may be created. Another challenge is to be able to adjust the shape in situ.

There is accordingly a need for improved methods and systems for providing minimally invasive, adjustably shaped, safe and efficient cryogenic cooling of tissues.

### SUMMARY OF THE INVENTION

An endovascular near critical fluid based cryoablation catheter for creating a continuous elliptical or oval shaped lesion in tissue has an elongated shaft and a distal treatment section. At least one fluid delivery tube extends through the distal treatment section to transport a near critical fluid towards the distal tip. The catheter further includes at least one fluid return tube extending through the distal treatment section to transport the near critical fluid away from the distal tip. When activated, a flow of near critical fluid is circulated through the at least one fluid delivery tube and the at least one fluid return tube to transfer heat from the target tissue to the distal treatment section of the catheter thereby creating the ovular continuous lesion in the tissue.

The distal tissue treatment section may be controllably deployed or articulated. In one embodiment, the distal treatment section has a constrained state, and an unconstrained state different than the constrained state. The unconstrained state has a curvature to match a particular anatomical curvature of a target tissue to be ablated.

In embodiments, the deployed shape of the distal treatment section assumes an ovular shape and is adapted to circumscribe multiple pulmonary vein entries including, for example, the left superior pulmonary vein entry and the right pulmonary vein entry.

In embodiments, the deployed shape of the distal treatment section is adjustable or deformable.

In embodiments, the deployed shape of the distal treatment section has an elliptical shape and a preferential bias. The preferential bias causes the major axis to be reduced prior to the minor axis when the distal treatment section is subjected to forces arising from tissue contact.

In embodiments, an elongate control member extends along the intermediate section of the catheter, and is secured to the distal tip. The elongate control member is in movable cooperation with the intermediate section and causes movement of the distal tip relative to the intermediate section.

At least one tubular energy delivery member extends from the intermediate section to the distal tip, and the tubular energy delivery member comprises a linear first configuration and a planar closed-curve second configuration substantially perpendicular to the linear first configuration.

In embodiments, the closed curve configuration has an eccentricity not equal to zero. Additionally, in embodiments, manipulation of the control member adjusts the eccentricity of the planar second configuration.

A flow of near critical fluid through the energy delivery member to transfer heat from the target tissue to the distal treatment section of the catheter creates the continuous lesion in the tissue.

In embodiments, the distal treatment section in a deployed configuration comprises a first closed curve (e.g., a leaf shaped curve) having a first center, and a second closed curve having a second center. The distance between the first center of the first closed curve and the second center of the second closed curve is adjustable to modify the shape of the distal treatment section to make better contact with the target tissue.

In embodiments, the deployed configuration comprises a first closed curve and a second closed curve in telescoping and rotatable cooperation with the first closed curve such that the first closed curve and second closed curve may be moved between a substantially concentric arrangement and an eccentric arrangement to modify the shape of the distal treatment section to make better contact with the target tissue.

In embodiments, the distal treatment section comprises a shape memory or superelastic material. A non-limiting exemplary superelastic material is Nitinol. In embodiments the fluid delivery tube and the fluid return tube comprises the superelastic material.

The diameter of the deployed shape may vary. In embodiments the deployed shape comprises a diameter ranging from 1 to 6 cm.

In embodiments, the distal treatment section has a preset shape to match a specific lesion to be created. The distal treatment section has a treatment shape adapted to create a lesion circumscribing the left superior and left inferior PV entries. In embodiments, the deployed treatment shape is substantially two dimensional and selected from the following: oval, heart, egg, butterfly, ellipse, FIG. 8, and clover

In embodiments, the distal treatment section includes a tube bundle formed of a plurality of fluid return tubes and one or more fluid delivery tubes.

In embodiments, an endovascular near critical fluid based cryoablation method for creating a continuous lesion in cardiac tissue comprises inserting a catheter comprising a distal treatment section into a patient's vasculature. The method further comprises the step of navigating the distal treatment section to the heart, and through an opening in the heart until the distal treatment section is within a space in the heart.

Exposing the distal treatment section of the elongate shaft by moving an outer sheath relative to the distal treatment section. The distal treatment section assuming a high profile intermediate configuration upon being unconstrained.

Adjusting the eccentricity of the intermediate shape into an ovular second shape.

Circulating a near critical fluid through at least one fluid delivery tube extending through the distal treatment section while the distal treatment section is in contact with a first target section of cardiac tissue.

In embodiments, the adjusting is performed by urging the distal section against the walls of tissue, and preferentially biasing a major axis to decrease prior to a minor axis.

In embodiments, the adjusting is performed by manipulation of a control wire fastened to the end of the energy delivery tubes.

In embodiments, the adjusting is performed by rotating a pair of circles away from one another until the second shape is formed, and wherein the second shape is one selected from the group consisting of a heart, oval, egg, clover, butterfly, and FIG. 8.

In embodiments, the catheter is navigated to a space within the left atrium, and the method further comprising advancing a guide sheath through the septum and into the left atrium thereby providing access to the first target section of cardiac tissue.

The method further comprising advancing a first guidewire through the guide sheath and into a first PV entry.

The method further comprising advancing a second guidewire through the guide sheath and into a second PV entry.

The method further comprising advancing the catheter simultaneously along the first and second guidewires towards the first and second PV entries, thereby centering the distal section of the catheter between the first and second PV entries.

The method wherein the first and second PV entries are the LSPV and LIPV entries respectively.

The method further comprising creating a single continuous oval-shaped lesion along the heart tissue enveloping both LSPV and the LIPV entries.

In embodiments, at least one of the fluid delivery tube and the fluid return tube comprises a superelastic material.

In embodiments the activation of the cooling is halted when a threshold condition is met. The threshold condition is preferably one condition selected from the group consisting of: length of lesion, thickness of lesion, time elapsed, energy transferred, temperature change, pressure change, flowrate change, and power change.

In embodiments the step of creating the lesion may be performed by creating the lesion having a length ranging from 2 to 10 cm. The lesion may be formed to have a thickness extending the entire thickness of a heart wall for the entire length of the distal treatment section of the catheter in contact with the heart wall.

In embodiments the method further comprises partially ejecting the distal treatment section from an outer sleeve, and observing a location of distal treatment section under an imaging modality prior to activation.

Additional embodiments of the present invention are directed to a cryoablation catheter for creating at least one lesion in tissue. The catheter comprises an elongate shaft having an intermediate section and a distal tip movable relative to the intermediate section, at least one elongate control member extending along the intermediate section and secured to the distal tip, the elongate control member being movable relative to the intermediate section for causing movement of the distal tip relative to the intermediate section, and at least one energy delivery member extending along the intermediate section to the distal tip, the at least one energy delivery member comprising a linear first configuration and an elliptical second configuration. Manipulation of the control member adjusts a shape of the at least one energy delivery member. The one energy delivery member can be a cryogen/fluid delivery tube.

Another embodiment is directed to an endovascular cryoablation catheter for creating at least one lesion in target tissue. The catheter comprises an elongate shaft having an intermediate section, a distal treatment section and at least one energy delivery member extending there through, where (i) the distal treatment section comprises a low-profile undeployed configuration and a high-profile substantially planar deployed configuration, and (ii) the deployed configuration comprises a first closed curve having a first center and a second closed curve having a second center. The catheter also includes a means to control movement of the first closed curve relative to the second closed curve such that a distance between the first center and the second center can be adjusted.

A further embodiment is directed to a endovascular cryoablation catheter for creating at least one continuous lesion in target tissue wherein the catheter comprises an elongate shaft having an intermediate section and a distal treatment section having at least one tubular energy delivery member extending there through. The distal treatment section comprises a low-profile undeployed configuration and a high-profile substantially planar deployed configuration, where the deployed configuration comprises a first leaf and a second leaf in telescoping and rotatable cooperation with the first leaf such that the first leaf and second leaf may be moved between a substantially concentric arrangement and an eccentric arrangement.

Embodiments are also directed to a method of creating a continuous lesion in cardiac tissue in a heart, where the method comprises inserting a catheter having an inner elongate shaft with a distal treatment section, at least one cryogen delivery tube and an outer sheath axially movable relative to the inner elongate shaft, into a patient's vasculature; navigating the distal treatment section of the catheter to the heart and through an opening in the heart until the distal treatment section is within a space in the heart; exposing the distal treatment section of the elongate shaft by moving the outer sheath relative to the distal treatment section; transforming the distal treatment section from a linear low profile first shape, to an intermediate shape, to a planar curved second shape, wherein the step of transforming comprises adjusting the eccentricity of the intermediate shape into the curved second shape; contacting the curved second shape with the cardiac tissue; and circulating a near critical fluid through the at least one cryogen delivery tube while the distal treatment section is in contact with the cardiac tissue.

Another embodiment is directed to a system for creating at least one lesion in target tissue. The system comprises a cryoablation catheter comprising (1) an elongate shaft having an intermediate section and a distal treatment section where the distal treatment section comprises a low-profile, undeployed configuration and a high-profile deployed configuration, where (i) the deployed configuration has an eccentric shape comprising a major axis and a minor axis less than the major axis, and (ii) the distal treatment section in the deployed configuration comprises a preferential bias such that the major axis is reduced prior to the minor axis when the distal treatment section is subjected to forces arising from contacting the tissue and (2) at least one energy delivery member extending along the elongate shaft. The system also includes a console for controlling a flow of cryogen to the at least one energy delivery member to transfer heat from the target tissue to the distal treatment section thereby creating the at least one lesion in the target tissue.

### BRIEF DESCRIPTION OF THE DRAWINGS

The description, objects and advantages of the present invention will become apparent from the detailed description to follow, together with the accompanying drawings wherein:

FIG. 1 illustrates a typical cryogen phase diagram;

FIG. 2A is a schematic illustration of a cryogenic cooling system;

FIG. 2B is a cryogen phase diagram to illustrate a method for cryogenic cooling;

FIG. 3 is a flow diagram of the cooling method of FIG. 2A;

FIG. 4 is a schematic illustration of a cryogenic generator;

FIG. 5 is a perspective view of a cryoprobe;

FIG. 6 is a view taken along line 6-6 of FIG. 5;

FIG. 7 is a perspective view of cryoprobe of FIG. 5 operated to generate an iceball;

FIG. 8 is a perspective view of the cryoprobe of FIG. 5 that is bent to approximately 180° to form a commensurately bent iceball;

FIG. 9 illustrates the cryoprobe sufficiently bent so as to form a loop;

FIG. 10 is a perspective view of another cryoprobe having a flexible distal section;

FIG. 11 is a view taken along line 11-11 of FIG. 10;

FIG. 12 is a side view of another cryoprobe including a handle having an inlet shaft and outlet shaft therein;

FIGS. 13-15 are schematic cross sectional views showing example alternative arrangements of fluid transfer tubes.

FIG. 16 is an illustration of a cryoablation system including a cryoablation catheter;

FIG. 17 is a partial perspective view of a cryoablation catheter having a curved distal treatment section;

FIG. 18 is an enlarged view of the proximal end of the distal treatment section shown in FIG. 17;

FIG. 19 is an enlarged view of the distal tip of the distal treatment section shown in FIG. 17;

FIGS. 20-23 are illustrations of a distal treatment section being deployed from a first configuration to a second configuration;

FIGS. 24-27 are illustrations of a distal treatment section being deployed from a constrained state, to a plurality of different shapes;

FIGS. 28-30 are illustrations of various distal deployed treatment sections having circular shapes;

FIGS. 31-33 are illustrations of various distal deployed treatment sections having elliptical shapes;

FIGS. 34a-34j are illustrations of a distal treatment section being deployed from an initial linear configuration, through a plurality of intermediate three dimensional configurations, and to a deployed substantially planar and elliptical configuration;

FIGS. 35a-35b are end top perspective views of a distal treatment section in a deployed configuration in a model tissue and being adjusted in shape to make greater contact with the surface of the model tissue;

FIGS. 36a-36b are side top perspective views of a distal treatment section in a deployed configuration in a model tissue and being adjusted in shape to make greater contact with the surface of the model tissue;

FIGS. 37-38 are perspective views of a handle portion of a cryoablation catheter;

FIG. 39 is an illustration of a heart, and locations of various target lesions;

FIG. 40 is an illustration of a endovascular catheterization to access the heart;

FIGS. 41-43 are illustrations of a procedure to place a distal section of a cryoablation catheter against the endocardial wall in the left atrium, circumscribing the left superior and inferior pulmonary vein entries; and

FIGS. 44-45 are illustrations of a procedure to place a distal section of a cryoablation catheter against the endocardial wall in the left atrium, circumscribing the right superior and inferior pulmonary vein entries.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Embodiments of the invention make use of thermodynamic processes using cryogens that provide cooling without encountering the phenomenon of vapor lock.

Cryogen Phase Diagram and Near Critical Point

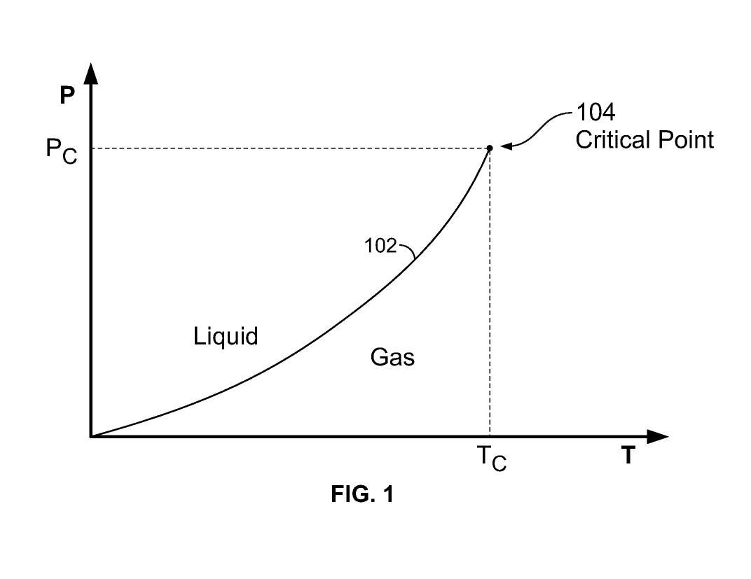

This application uses phase diagrams to illustrate and compare various thermodynamic processes. An example phase diagram is shown in FIG. 1. The axes of the diagram correspond to pressure P and temperature T, and includes a phase line 102 that delineates the locus of all (P, T) points where liquid and gas coexist. For (P, T) values to the left of the phase line 102, the cryogen is in a liquid state, generally achieved with higher pressures and lower temperatures, while (P, T) values to the right of the phase line 102 define regions where the cryogen is in a gaseous state, generally achieved with lower pressures and higher temperatures. The phase line 102 ends abruptly in a single point known as the critical point 104. In the case of nitrogen N₂, the critical point is at P_{c}=33.94 bar and T_{c}=−147.15° C.

When a fluid has both liquid and gas phases present during a gradual increase in pressure, the system moves up along the liquid-gas phase line 102. In the case of N₂, the liquid at low pressures is up to two hundred times more dense than the gas phase. A continual increase in pressure causes the density of the liquid to decrease and the density of the gas phase to increase, until they are exactly equal only at the critical point 104. The distinction between liquid and gas disappears at the critical point 104. The blockage of forward flow by gas expanding ahead of the liquid cryogen is thus avoided by conditions surrounding the critical point, defined herein as “near-critical conditions.” Factors that allow greater departure from the critical point while maintaining a functional flow include greater speed of cryogen flow, larger diameter of the flow lumen and lower heat load upon the thermal exchanger, or cryoprobe tip.

As the critical point is approached from below, the vapor phase density increases and the liquid phase density decreases until right at the critical point, where the densities of these two phases are exactly equal. Above the critical point, the distinction of liquid and vapor phases vanishes, leaving only a single, supercritical phase. All gases obey quite well the following van der Waals equation of state:

(p+3/v²)(3v−1)=8t [Eq. 1]

where p=P/P_{c}, v=V/V_{c}, and t=T/T_{c}, and P_{c}, V_{c}, and T_{c} are the critical pressure, critical molar volume, and the critical temperature respectively.

The variables v, p, and t are often referred to as the “reduced molar volume,” the “reduced pressure,” and the “reduced temperature,” respectively. Hence, any two substances with the same values of p, v, and t are in the same thermodynamic state of fluid near its critical point. Eq. 1 is thus referred to as embodying the “Law of Corresponding States.” This is described more fully in H. E. Stanley, Introduction to Phase Transitions and Critical Phenomena (Oxford Science Publications, 1971), the entire disclosure of which is incorporated herein by reference for all purposes. Rearranging Eq. 1 provides the following expression for v as a function of p and t:

pv³−(p+8t)v²+9v−3=0. [Eq. 2]

The reduced molar volume of the fluid v may thus be thought of as being an exact function of only the reduced pressure t and the reduced pressure p.

Typically, in embodiments of the invention, the reduced pressure p is fixed at a constant value of approximately one, and hence at a fixed physical pressure near the critical pressure, while the reduced temperature t varies with the heat load applied to the needle. If the reduced pressure p is a constant set by the engineering of the system, then the reduced molar volume v is an exact function of the reduced temperature t. In embodiments of the invention, the needle's operating pressure p may be adjusted so that over the course of variations in the temperature t of the needle, v is maintained below some maximum value at which the vapor lock condition will result. It is generally desirable to maintain p at the lowest value at which this is true since boosting the pressure to achieve higher values of p may involve use of a more complex and more expensive compressor, resulting in more expensive procurement and maintenance of the entire needle support system and lower overall wall plug efficiency. As used herein, “wall plug efficiency” refers to the total cooling power of the apparatus divided by the power obtained from a line to operate the system.

The conditions that need to be placed on v depend in a complex and non-analytic way on the volume flow rate dV/dt, the heat capacity of the liquid and vapor phases, and the transport properties such as the thermal conductivity, viscosity, etc., in both the liquid and the vapor. This exact relationship cannot be derived in closed form algebraically, but may be determined numerically by integrating the model equations that describe mass and heat transport within the needle. Conceptually, vapor lock occurs when the rate of heating of the needle produces the vapor phase, and when the cooling power of this vapor phase, which is proportional to the flow rate of the vapor times its heat capacity divided by its molar volume, is not able to keep up with the rate of heating to the needle. When this occurs, more and more of the vapor phase is formed in order to absorb the excess heat through the conversion of the liquid phase to vapor in the cryogen flow. This creates a runaway condition where the liquid converts into vapor phase to fill the needle, and effectively all cryogen flow stops due to the large pressure that results in this vapor phase as the heat flow into the needle increases its temperature and pressure rapidly. This condition is called “vapor lock.” Since the liquid and vapor phases are identical in their molar volume, and hence cooling power at the critical point, the cooling system at or above the critical point can never vapor lock. But for conditions slightly below the critical below the critical point, the needle may avoid vapor lock as well.

Embodiments of the invention avoid the occurrence of vapor lock and permit decreased probe sizes by operating in cryogen pressure-temperature regimes that avoid any crossing of the liquid-gas phase line. In particular embodiments, cryogenic cooling is achieved by operating near the critical point for the cryogen. When operating in this region, heat flows into the near-critical cryogen from the surrounding environment since the critical-point temperature (e.g., −147° C. in the case of N₂) is much colder that the surrounding environment. This heat is removed by the flow of the near critical cryogen through the tip of a cryoprobe, even though there is no latent heat of evaporation to assist with the cooling process. While the scope of the invention is intended to include operation in any regime having a pressure greater than the critical-point pressure, the cooling efficiency tends to decrease as the pressure is increased above the critical pressure. This is a consequence of increasing energy requirements needed to achieve flow at higher operating pressures.

Cryoablation Systems

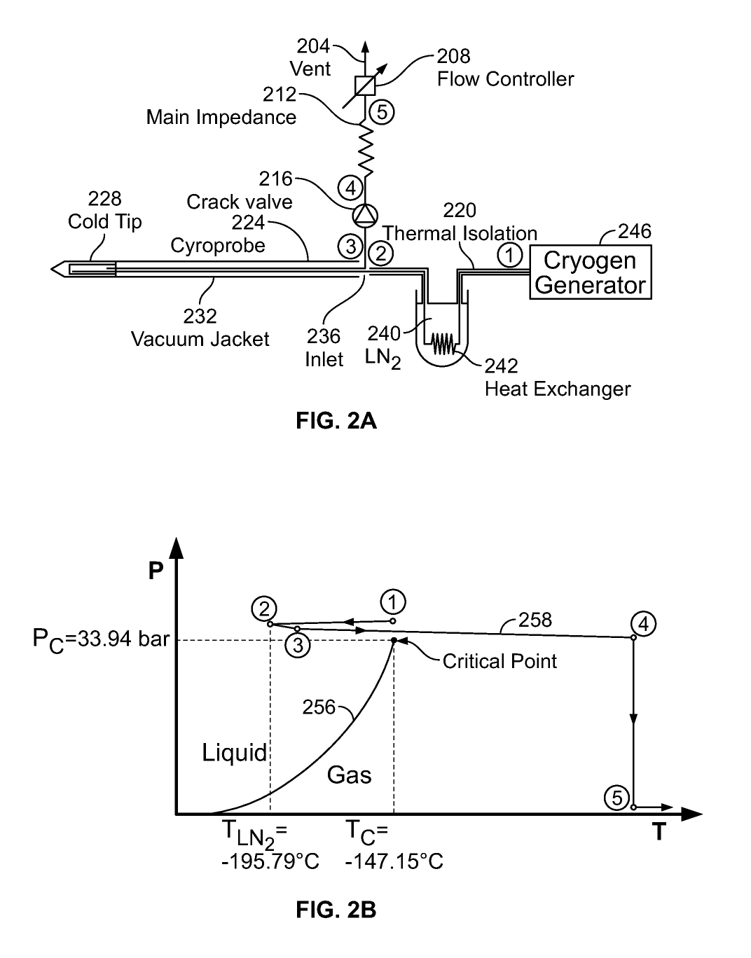

FIG. 2A provides a schematic illustration of a structural arrangement for a cryogenic system in one embodiment, and FIG. 2B provides a phase diagram that illustrates a thermodynamic path taken by the cryogen when the system of FIG. 2A is operated. The circled numerical identifiers in the two figures correspond so that a physical position is indicated in FIG. 2A where operating points identified along the thermodynamic path are achieved. The following description thus sometimes makes simultaneous reference to both the structural drawing of FIG. 2A and to the phase diagram of FIG. 2B in describing physical and thermodynamic aspects of the cooling flow. For purposes of illustration, both FIGS. 2A and 2B make specific reference to a nitrogen cryogen, but this is not intended to be limiting. The invention may more generally be used with any suitable cryogen, as will be understood by those of skill in the art; merely by way of example, alternative cryogens that may be used include argon, helium, hydrogen, and oxygen. In FIG. 2B, the liquid-gas phase line is identified with reference label 256 and the thermodynamic path followed by the cryogen is identified with reference label 258.

A cryogenic generator 246 is used to supply the cryogen at a pressure that exceeds the critical-point pressure P_{c} for the cryogen at its outlet, referenced in FIGS. 2A and 2B by label \textcircled{1}. The cooling cycle may generally begin at any point in the phase diagram having a pressure above or slightly below P_{c}, although it is advantageous for the pressure to be near the critical-point pressure P_{c}. The cooling efficiency of the process described herein is generally greater when the initial pressure is near the critical-point pressure P_{c} so that at higher pressures there may be increased energy requirements to achieve the desired flow. Thus, embodiments may sometimes incorporate various higher upper boundary pressure but generally begin near the critical point, such as between 0.8 and 1.2 times P_{c}, and in one embodiment at about 0.85 times P_{c}.

As used herein, the term “near critical” refers to near the liquid-vapor critical point. Use of this term is equivalent to “near a critical point” and it is the region where the liquid-vapor system is adequately close to the critical point, where the dynamic viscosity of the fluid is close to that of a normal gas and much less than that of the liquid; yet, at the same time its density is close to that of a normal liquid state. The thermal capacity of the near critical fluid is even greater than that of its liquid phase. The combination of gas-like viscosity, liquid-like density and very large thermal capacity makes it a very efficient cooling agent. In other words, reference to a near critical point refers to the region where the liquid-vapor system is adequately close to the critical point so that the fluctuations of the liquid and vapor phases are large enough to create a large enhancement of the heat capacity over its background value. The near critical temperature is a temperature within ±10% of the critical point temperature. The near critical pressure is between 0.8 and 1.2 times the critical point pressure.

Referring again to FIG. 2A, the cryogen is flowed through a tube, at least part of which is surrounded by a reservoir 240 of the cryogen in a liquid state, reducing its temperature without substantially changing its pressure. In FIG. 2A, reservoir is shown as liquid N₂, with a heat exchanger 242 provided within the reservoir 240 to extract heat from the flowing cryogen. Outside the reservoir 240, thermal insulation 220 may be provided around the tube to prevent unwanted warming of the cryogen as it is flowed from the cryogen generator 246. At point \textcircled{2}, after being cooled by being brought into thermal contact with the liquid cryogen, the cryogen has a lower temperature but is at substantially the initial pressure. In some instances, there may be a pressure change, as is indicated in FIG. 2B in the form of a slight pressure decrease, provided that the pressure does not drop substantially below the critical-point pressure P_{c}, i.e. does not drop below the determined minimum pressure. In the example shown in FIG. 2B, the temperature drop as a result of flowing through the liquid cryogen is about 47° C.

The cryogen is then provided to a device for use in cryogenic applications. In the exemplary embodiment shown in FIG. 2A, the cryogen is provided to an inlet 236 of a cryoprobe 224, such as may be used in medical cryogenic applications, but this is not a requirement.

In embodiments, the cryogen may be introduced through a proximal portion of a catheter, along a flexible intermediate section of the catheter, and into the distal treatment section of the catheter. At the point when the cryogen is provided to such treatment region of the device, indicated by label \textcircled{2 and 3} in FIGS. 2A and 2B, there may be a slight change in pressure and/or temperature of the cryogen as it moves through an interface with the device, i.e. such as when it is provided from the tube to the cryoprobe inlet 236 in FIG. 2A. Such changes may typically show a slight increase in temperature and a slight decrease in pressure. Provided the cryogen pressure remains above the determined minimum pressure (and associated conditions), slight increases in temperature do not significantly affect performance because the cryogen simply moves back towards the critical point without encountering the liquid-gas phase line 256, thereby avoiding vapor lock.

Thermal insulation along the shaft of the cryotherapy apparatus (e.g., needles), and along the support system that delivers near-critical freeze capability to these needles, may use a vacuum of better than one part per million of atmospheric pressure. Such a vacuum may not be achieved by conventional two-stage roughing pumps alone. The percutaneous cryotherapy system in an embodiment thus incorporates a simplified method of absorption pumping rather than using expensive and maintenance-intensive high-vacuum pumps, such as diffusion pumps or turbomolecular pumps. This may be done on an internal system reservoir of charcoal, as well as being built into each individual disposable probe.

Embodiments incorporate a method of absorption pumping in which the liquid nitrogen bath that is used to sub-cool the stream of incoming nitrogen near its critical point is also used to cool a small volume of clean charcoal. The vast surface area of the charcoal permits it to absorb most residual gas atoms, thus lowering the ambient pressure within its volume to well below the vacuum that is used to thermally insulate the needle shaft and the associated support hardware. This volume that contains the cold charcoal is attached through small-diameter tubing to the space that insulates the near-critical cryogen flow to the needles. Depending upon the system design requirements for each clinical use, the charcoal may be incorporated into the cooling reservoir of liquid cryogen 240 seen in FIG. 2A, or become part of the cryoprobe 224, near the connection of the extension hose near the inlet 236. Attachments may be made through a thermal contraction bayonet mount to the vacuum space between the outer shaft of the vacuum jacketed needles and the internal capillaries that carry the near-critical cryogen, and which is thermally insulated from the surrounding tissue. In this manner, the scalability of the system extends from simple design constructions, whereby the charcoal-vacuum concept may be incorporated into smaller reservoirs where it may be more convenient to draw the vacuum. Alternatively, it may be desirable for multiple-probe systems to individually incorporate small charcoal packages into each cryoprobe near the junction of the extension close/cryoprobe with the machine interface 236, such that each hose and cryoprobe draws its own vacuum, thereby further reducing construction costs.

Flow of the cryogen from the cryogen generator 246 through the cryoprobe 224 or other device may be controlled in the illustrated embodiment with an assembly that includes a crack valve 216, a flow impedance, and a flow controller. The cryoprobe 224 itself may comprise a vacuum jacket 232 along its length and may have a cold tip 228 that is used for the cryogenic applications. Unlike a Joule-Thomson probe, where the pressure of the working cryogen changes significantly at the probe tip, these embodiments of the invention provide relatively little change in pressure throughout the probe. Thus, at point \textcircled{4}, the temperature of the cryogen has increased approximately to ambient temperature, but the pressure remains elevated. By maintaining the pressure above the critical-point pressure P_{c} throughout the process, the liquid-gas phase line 256 is never encountered along the thermodynamic path 258 and vapor lock is thereby avoided. The cryogen pressure returns to ambient pressure at point \textcircled{5} before passing through the flow controller 208, which is typically located well away from the cryoprobe 224. The cryogen may then be vented through vent 204 at substantially ambient conditions. See also U.S. Pat. No. 8,387,402 to Littrup et al. for arrangements of near critical fluid cryoablation systems.

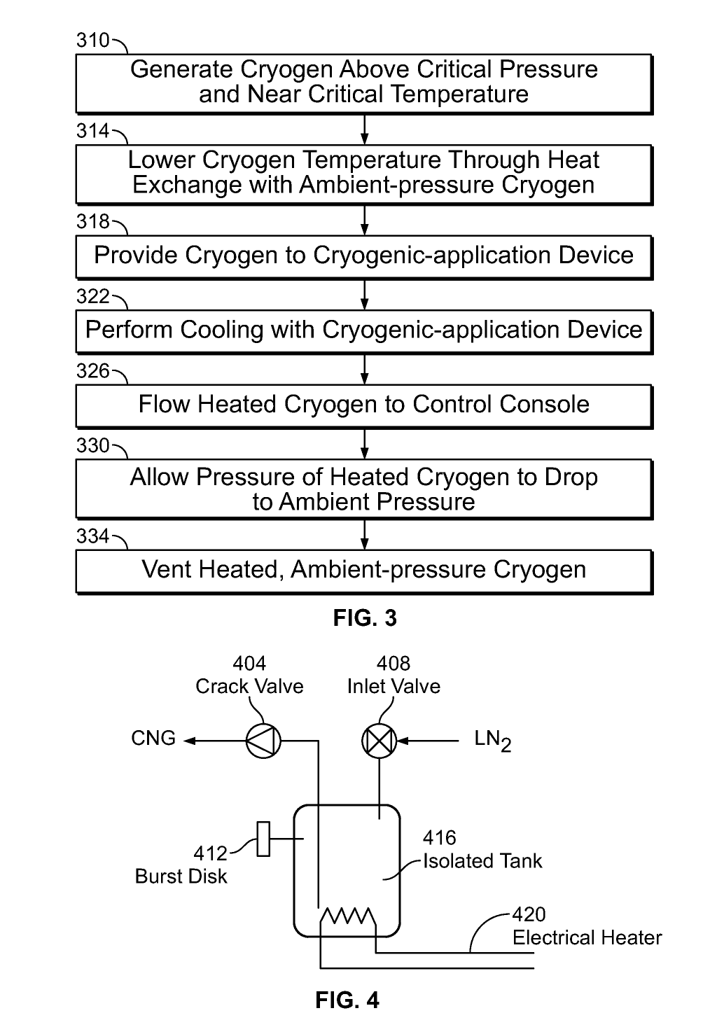

A method for cooling in one embodiment in which the cryogen follows the thermodynamic path shown in FIG. 2B is illustrated with the flow diagram of FIG. 3. At block 310, the cryogen is generated with a pressure that exceeds the critical-point pressure and is near the critical-point temperature. The temperature of the generated cryogen is lowered at block 314 through heat exchange with a substance having a lower temperature. In some instances, this may conveniently be performed by using heat exchange with an ambient-pressure liquid state of the cryogen, although the heat exchange may be performed under other conditions in different embodiments. For instance, a different cryogen might be used in some embodiments, such as by providing heat exchange with liquid nitrogen when the working fluid is argon. Also, in other alternative embodiments, heat exchange may be performed with a cryogen that is at a pressure that differs from ambient pressure, such as by providing the cryogen at lower pressure to create a colder ambient.

The further cooled cryogen is provided at block 318 to a cryogenic-application device, which may be used for a cooling application at block 322. The cooling application may comprise chilling and/or freezing, depending on whether an object is frozen with the cooling application. The temperature of the cryogen is increased as a result of the cryogen application, and the heated cryogen is flowed to a control console at block 326. While there may be some variation, the cryogen pressure is generally maintained greater than the critical-point pressure throughout blocks 310-326; the principal change in thermodynamic properties of the cryogen at these stages is its temperature. At block 330, the pressure of the heated cryogen is then allowed to drop to ambient pressure so that the cryogen may be vented, or recycled, at block 334. In other embodiments, the remaining pressurized cryogen at block 326 may also return along a path to block 310 to recycle rather than vent the cryogen at ambient pressure.

Cryogen Generators

There are a variety of different designs that may be used for the cryogen source or generator 246 in providing cryogen at a pressure that exceeds the critical-point pressure, or meets the near-critical flow criteria, to provide substantially uninterrupted cryogen flow at a pressure and temperature near its critical point. In describing examples of such designs, nitrogen is again discussed for purposes of illustration, it being understood that alternative cryogens may be used in various alternative embodiments. FIG. 4 provides a schematic illustration of a structure that may be used in one embodiment for the cryogen generator. A thermally insulated tank 416 has an inlet valve 408 that may be opened to fill the tank 416 with ambient liquid cryogen. A resistive heating element 420 is located within the tank 416, such as in a bottom section of the tank 416, and is used to heat the cryogen when the inlet valve is closed. Heat is applied until the desired operating point is achieved, i.e. at a pressure that exceeds the near-critical flow criteria. A crack valve 404 is attached to an outlet of the tank 416 and set to open at the desired pressure. In one embodiment that uses nitrogen as a cryogen, for instance, the crack valve 404 is set to open at a pressure of about 33.9 bar, about 1 bar greater than the critical-point pressure. Once the crack valve 404 opens, a flow of cryogen is supplied to the system as described in connection with FIGS. 2A and 2B above.

A burst disk 412 may also be provided consistent with safe engineering practices to accommodate the high cryogen pressures that may be generated. The extent of safety components may also depend in part on what cryogen is to be used since they have different critical points. In some instances, a greater number of burst disks and/or check valves may be installed to relieve pressures before they reach design limits of the tank 416 in the event that runaway processes develop.

During typical operation of the cryogen generator, an electronic feedback controller maintains current through the resistive heater 420 to a level sufficient to produce a desired flow rate of high-pressure cryogen into the system. The actual flow of the cryogen out of the system may be controlled by a mechanical flow controller 208 at the end of the flow path as indicated in connection with FIG. 2A. The amount of heat energy needed to reach the desired cryogen pressures is typically constant once the inlet valve 408 has been closed. The power dissipated in the resistive heater 420 may then be adjusted to keep positive control on the mechanical flow controller 208. In an alternative embodiment, the mechanical flow controller 208 is replaced with the heater controller for the cryogen generator. In such an embodiment, once the crack valve 404 opens and high-pressure cryogen is delivered to the rest of the system, the feedback controller continuously adjusts the current through the resistive heater to maintain a desired rate of flow of gaseous cryogen out of the system. The feedback controller may thus comprise a computational element to which the heater current supply and flow controller are interfaced.

In embodiments, a cryogen tank comprising a high pressure cryogen is provided. Alternatively, a gas line from the wall may supply the high pressure cryogen.

Flexible Multi-Tubular Cryoablation Catheter

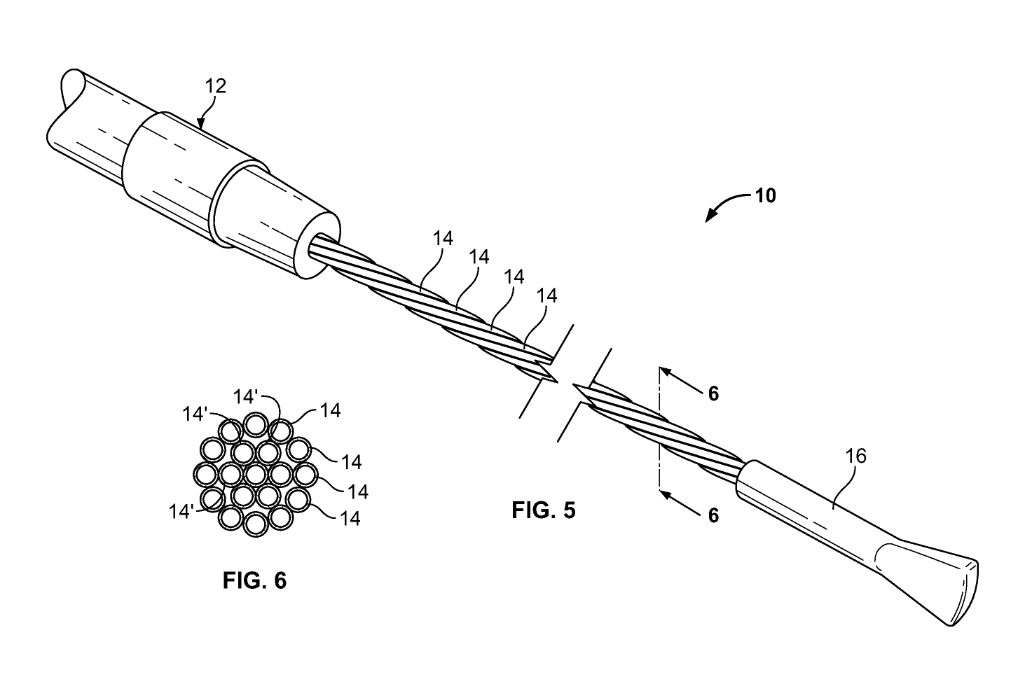

FIGS. 5 and 6 illustrate a flexible multi-tubular cryoprobe 10. The cryoprobe 10 includes a housing 12 for receiving an inlet flow of near critical cryogenic fluid from a fluid source (not shown) and for discharging an outlet flow of the cryogenic fluid. A plurality of fluid transfer tubes 14, 14′ are securely attached to the housing 12. These tubes include a set of inlet fluid transfer tubes 14 for receiving the inlet flow from the housing; and, a set of outlet fluid transfer tubes 14′ for discharging the outlet flow to the housing 12. Each of the fluid transfer tubes 14, 14′ is formed of material that maintains flexibility in a full range of temperatures from −200° C. to ambient temperature. Each fluid transfer tube has an inside diameter in a range of between about 0.10 mm and 1.0 mm (preferably between about 0.20 mm and 0.50 mm). Each fluid transfer tube has a wall thickness in a range of between about 0.01 mm and 0.30 mm (preferably between about 0.02 mm and 0.10 mm). An end cap 16 is positioned at the ends of the fluid transfer tubes 14, 14′ to provide fluid transfer from the inlet fluid transfer tubes 14 to the outlet fluid transfer tubes 14′.

In embodiments the tubes 14, 14′ are formed of annealed stainless steel or a polyimide, preferably Kapton® polyimide. These materials maintain flexibility at a near critical temperature. By flexibility, it is meant the ability of the cryoprobe to be bent in the orientation desired by the user without applying excess force and without fracturing or resulting in significant performance degradation.

The cryogenic fluid utilized is preferably near critical nitrogen. However, other fluids may be utilized such as argon, neon, helium or others.

The fluid source for the cryogenic fluid may be provided from a suitable mechanical pump or a non-mechanical critical cryogen generator as described above. Such fluid sources are disclosed in, for example, U.S. patent application Ser. No. 10/757,768 which issued as U.S. Pat. No. 7,410,484, on Aug. 12, 2008 entitled “CRYOTHERAPY PROBE”, filed Jan. 14, 2004 by Peter J. Littrup et al.; U.S. patent application Ser. No. 10/757,769 which issued as U.S. Pat. No. 7,083,612 on Aug. 1, 2006, entitled “CRYOTHERAPY SYSTEM”, filed Jan. 14, 2004 by Peter J. Littrup et al.; U.S. patent application Ser. No. 10/952,531 which issued as U.S. Pat. No. 7,273,479 on Sep. 25, 2007 entitled “METHODS AND SYSTEMS FOR CRYOGENIC COOLING” filed Sep. 27, 2004 by Peter J. Littrup et al. U.S. Pat. No. 7,410,484, U.S. Pat. No. 7,083,612 and U.S. Pat. No. 7,273,479 are incorporated herein by reference, in their entireties, for all purposes.

The endcap 16 may be any suitable element for providing fluid transfer from the inlet fluid transfer tubes to the outlet fluid transfer tubes. For example, endcap 16 may define an internal chamber, cavity, or passage serving to fluidly connect tubes 14, 14′.

There are many configurations for tube arrangements. In one class of embodiments the tubes are formed of a circular array, wherein the set of inlet fluid transfer tubes comprises at least one inlet fluid transfer tube defining a central region of a circle and wherein the set of outlet fluid transfer tubes comprises a plurality of outlet fluid transfer tubes spaced about the central region in a circular pattern. In the configuration shown in FIG. 6, the tubes 14, 14′ fall within this class of embodiments.

During operation, the cryogen fluid arrives at the cryoprobe through a supply line from a suitable nitrogen source at a temperature close to −200° C., is circulated through the multi-tubular freezing zone provided by the exposed fluid transfer tubes, and returns to the housing.

In embodiments, the nitrogen flow does not form gaseous bubbles inside the small diameter tubes under any heat load, so as to not create a vapor lock that limits the flow and the cooling power. By operating at the near critical condition the vapor lock is eliminated as the distinction between the liquid and gaseous phases disappears.

Embodiments of the present invention provides a substantial increase in the heat exchange area between the cryogen and tissue, over prior art cryoprobes, by this multi-tubular design. Depending on the number of tubes used, the present cryoprobes can increase the contact area several times over previous cryoprobes having similarly sized diameters with single shafts.

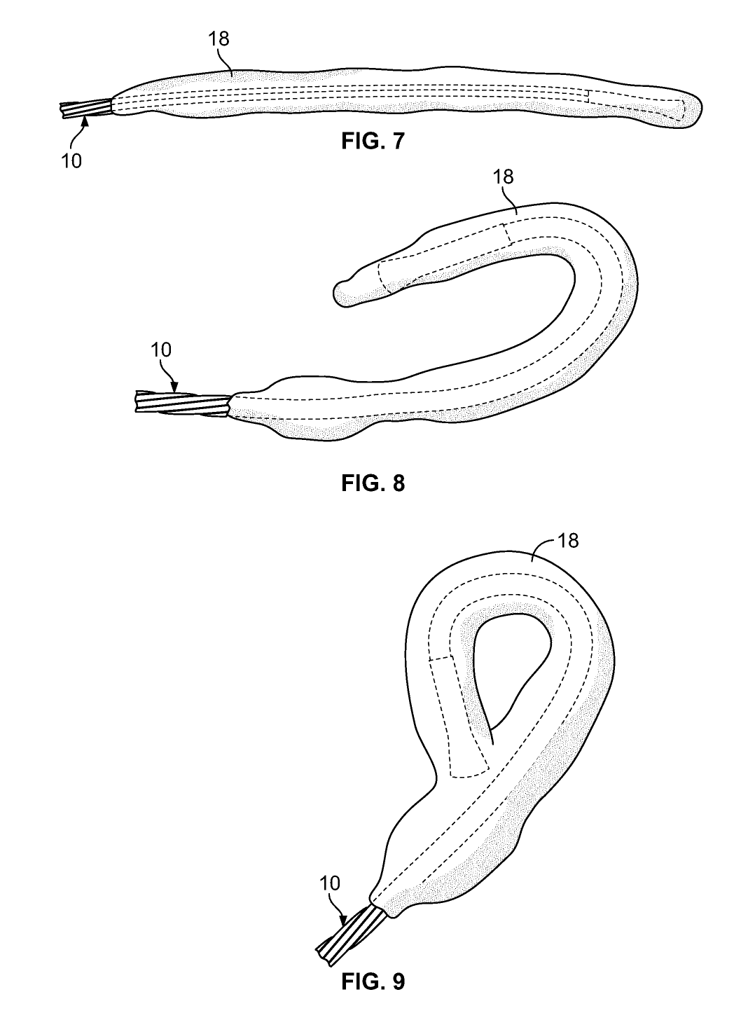

As can be seen in FIG. 7, an iceball 18 is generated about the cryoprobe 10. Referring now to FIG. 8, it can be seen that an iceball 18 can be created in the desired shape by bending or articulating the cryoprobe in the desired orientation. A complete iceball 18 loop can be formed, as shown in FIG. 9.

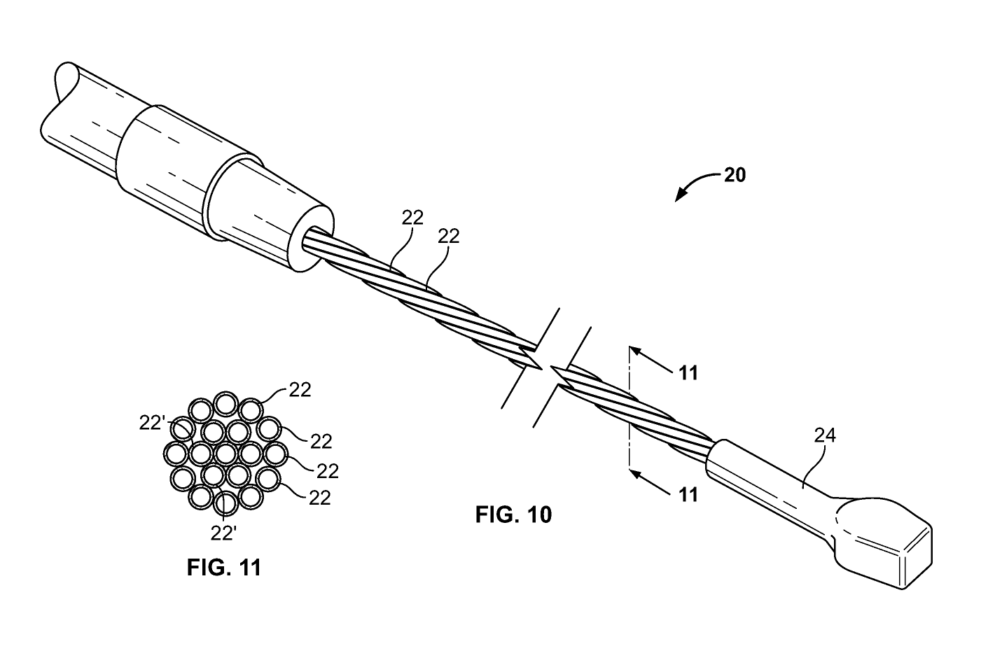

Referring now to FIG. 10, a cryoprobe 20 is illustrated, which is similar to the embodiment of FIG. 5, however, with this embodiment a polyimide material is used to form the tubes 22, 22′. Furthermore, this figure illustrates the use of a clamp 24 as an endcap. Although polyimide tubing is described to achieve flexibility and conformability to target structures, in other embodiments, as described further herein, the catheter may incorporate memory or shape set components to cause predetermined bends. Additionally, pull wires, actuators, and spine elements may be added to the distal section to create desirable bends and shapes.

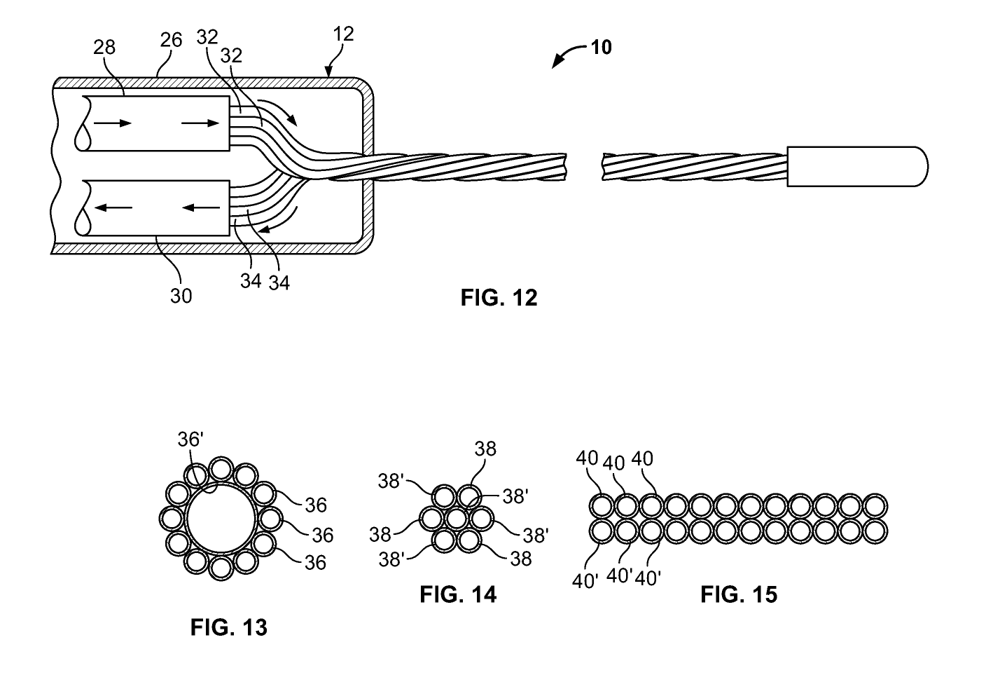

Referring now to FIG. 12, one embodiment of the housing 12 of a cryoprobe 10 is illustrated. The housing 12 includes a handle 26 that supports an inlet shaft 28 and an outlet shaft 30. The inlet shaft 28 is supported within the handle 26 for containing proximal portions of the set of inlet fluid transfer tubes 32. The outlet shaft 30 is supported within the handle 26 for containing proximal portions of the set of outlet fluid transfer tubes 34. Both of the shafts 28, 30 include some type of thermal insulation, preferably a vacuum, to isolate them.

Referring now to FIGS. 13-15 various configurations of tube configurations are illustrated. In FIG. 13 a configuration is illustrated in which twelve inlet fluid transfer tubes 36 circumscribe a single relatively large outlet fluid transfer tube 36′. In FIG. 14, three inlet fluid transfer tubes 38 are utilized with four outlet fluid transfer tubes 38′. In FIG. 15, a plane of inlet fluid transfer tubes 40 are formed adjacent to a plane of outlet of fluid transfer tubes 40′.

In an example, an annealed stainless steel cryoprobe was utilized with twelve fluid transfer tubes. There were six inlet fluid transfer tubes in the outer circumference and six outlet fluid transfer tubes in the center. The tubes were braided as shown in FIG. 5. The length of the freeze zone was 6.5 inches. Each fluid transfer tube had an outside diameter of 0.16 inch and an inside diameter 0.010 inch. The diameter of the resultant array of tubes was 0.075 inch. After a one minute freeze in 22° C. water and near-critical (500 psig) nitrogen flow of approximately 20 STP 1/min, ice covered the entire freeze zone of the flexible cryoprobe with an average diameter of about 0.55 inch. After four minutes the diameter was close to 0.8 inch. The warm cryoprobe could be easily bent to any shape including a full loop of approximately 2 inch in diameter without any noticeable change in its cooling power.

In another example, a polyimide cryoprobe was utilized with twenty-one fluid transfer tubes. There were ten inlet fluid transfer tubes in the outer circumference and eleven outlet fluid transfer tubes in the center. The tubes were braided. The length of the freeze zone was 6.0 inches. Each fluid transfer tube had an outside diameter of 0.0104 inch and an inside diameter 0.0085 inch. Each tube was pressure rated for about 1900 psig (working pressure 500 psig). The average diameter of the flexible portion of the cryoprobe was 1.15 mm (0.045 inch). The cryoprobe was extremely flexible with no perceivable “memory” in it. It bent by its own weight of just 1 gram and easily assumed any shape with a bending radius as little as 0.1 inch, including a 1 inch diameter “knot”. A full loop was created with the cryoprobe. After a one minute freeze in 22° C. water and near critical (500 psig) nitrogen flow of approximately 20 STP 1/min, ice covered the entire freeze zone of the flexible cryoprobe with an average diameter of 0.65 inch and in two minutes it closed the entire 1 inch hole inside the loop. See also, U.S. Publication No. 2011/0040297 to Babkin et al. for additional cryoprobe and catheter designs.

Cryoablation Catheter with Spring-Biased Distal Treatment Section

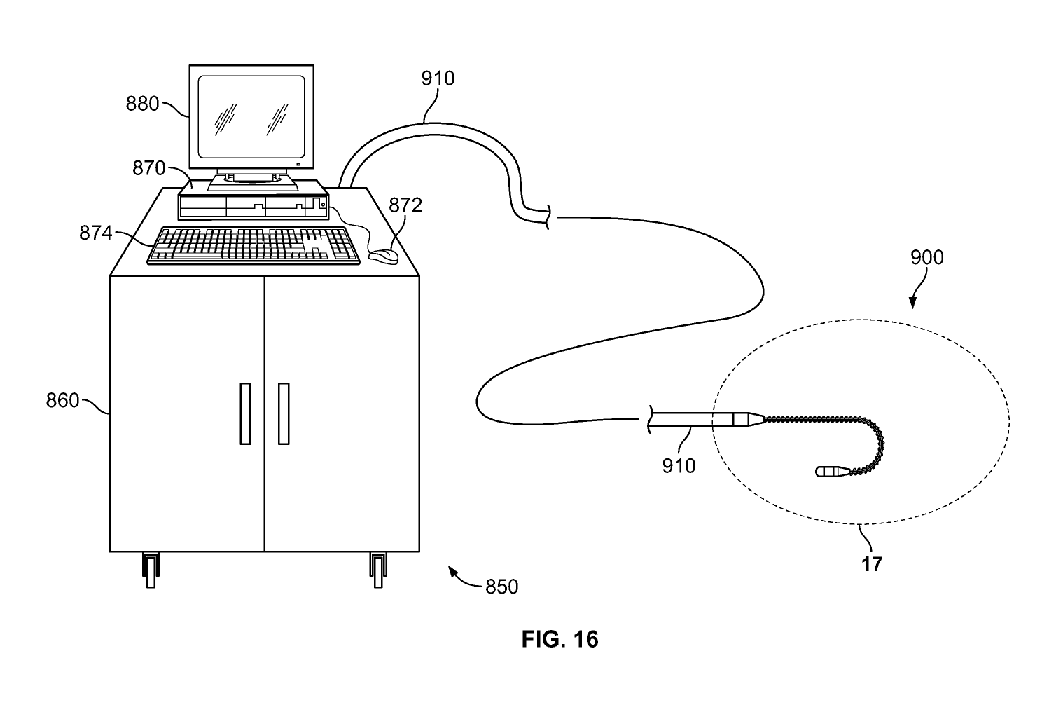

FIG. 16 illustrates a cryoablation system 850 having a cart or console 860 and a cryoablation catheter 900 detachably connected to the console via a flexible elongate tube 910. The cryoablation catheter 900, which shall be described in more detail below in connection with FIG. 17, includes a spring biased distal treatment section which serves to match the contour of a target anatomical region.

The console 860 may include a variety of components (not shown) such as, for example, a generator, controller, tank, valve, pump, etc. A computer 870 and display 880 are shown in FIG. 16 positioned on top of cart for convenient user operation. Computer may include a controller, or communicate with an external controller to drive components of the cryoablation systems such as a pump, valve or generator. Input devices such as a mouse 872 and a keyboard 874 may be provided to allow the user to input data and control the cryoablation devices.

In embodiments computer 870 is configured or programmed to control cryogen flowrate, pressure, and temperatures as described herein. Target values and real time measurement may be sent to, and shown, on the display 880.

With reference to FIG. 17 the distal treatment section 1010 is shown in a deflected or curved configuration and includes a proximal end 1012, a distal end 1014, and treatment or freeze zone 1016 therebetween. As will be described in more detail herein, the curvature of the treatment section may be controlled to match a particular anatomy such as the interior surface of the heart.

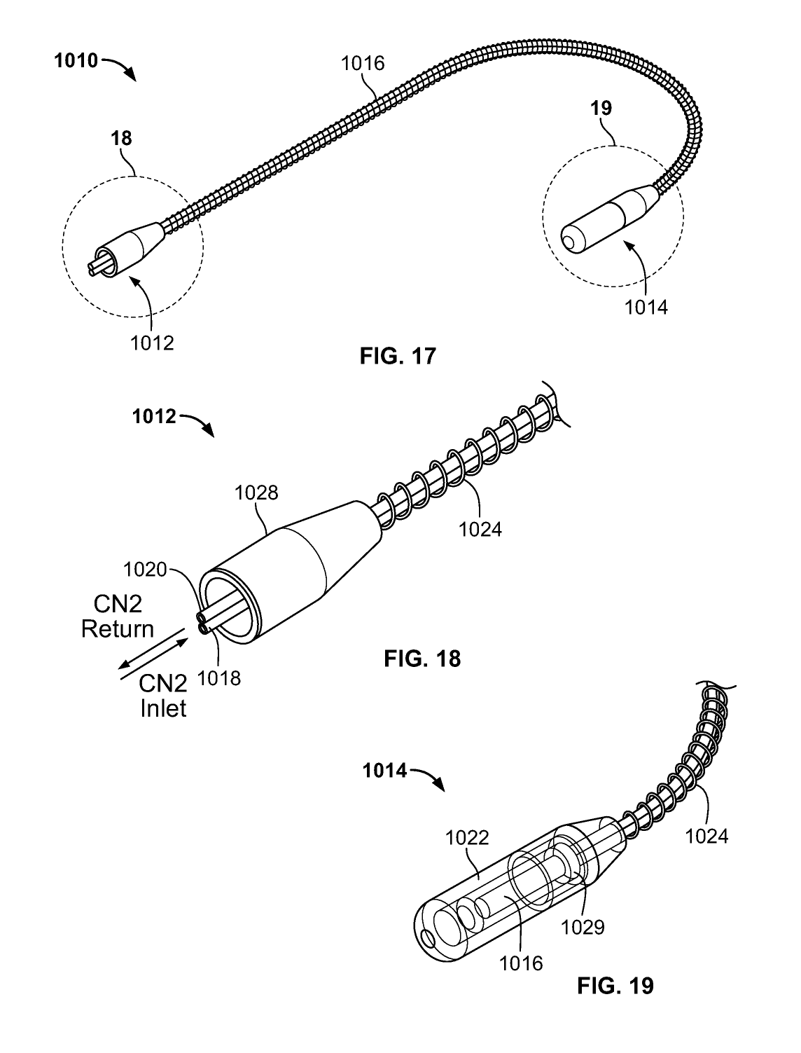

With reference to FIGS. 18 and 19 which show enlarged views of the proximal end 1012 and the distal end 1014 respectively, at least one fluid delivery tube 1018 extends through the distal treatment section to a chamber or cavity 1016 in the distal tip. A fluid return tube 1020 extends through the distal treatment section from the chamber 1016 to transport the cooling fluid from the chamber to a storage tank or exhaust structure as desired. As described herein, a cooling fluid may be transported from a fluid source, through an intermediate section of the catheter or apparatus, and through the tube bundle in order to freeze the target tissue placed in contact with the distal treatment section 1016.

The fluid transport tubes 1018,1020 in the treatment section are preferably made of a material adapted to safely hold fluids under pressure 2-3 times the working pressure. Consequently, secondary or redundant outer balloons/covers are unnecessary. Additionally, the tubes are desirably good thermal conductors in order to transfer heat from the tissue to the fluid. The fluid transport tubes 1018, 1020 preferably have an outer diameter ranging from 0.2 to 2 mm. The fluid transport tubes are shown being smooth, and without corrugations or grooves. However, in embodiments, the structures may include textures, ridges, and corrugations.

Additionally, in embodiments, the tubes are preferably made of materials that have a preset shape as described further herein. An exemplary material is a shape memory metal or alloy (e.g., Nitinol). However, other materials may be suitable including various polymers, stainless steels, spring steel, etc.

Attachment of the distal tip section to the body or intermediate section of the cryoablation catheter may be carried out as described herein and include, for example, a seal or transition hub 1028 which engages the outside of the intermediate section of the catheter (not shown). For example, with reference to FIG. 16, a hub may be joined to inlet line 910 of system 850. Glues, adhesives, and shrink tube sleeves may be incorporated into the designs to hold the components together. Insulation layers including an air or vacuum gap may be incorporated into the intermediate section of the catheter as described herein.

With reference to FIG. 19, the distal tip 1014 may include a seal and adhesive layers to secure the chamber to the plurality of transport tubes and to prevent leaks. The cap may include a redundant or double seals. For example, a second cap 1022 may be situated or encapsulate a first cap 1029. In this manner, a cooling liquid under the pressures described herein may be safely transported to and from the distal tip without the danger of a leak.

FIGS. 18-19 also show a tubular member 1024 surrounding the transport tubes. The tubular member 1024 maintains the transport tube bundle together when the treatment section is articulated or bends. The coil 1024 also allows tissue and bodily fluids to contact the transport tubes directly thereby increasing thermal conductivity between the cooling fluid and the target tissue. Although a coil is shown, the invention is not so limited and the coil need not be present. Alternative structures may be utilized to hold the tube bundle together so that it may actuated as a unit. Examples include tacking structures, welds, adhesives, two or more spot welds, and bands. Alternatively, tube elements may coextruded or formed to operate as an integrated articulatable member.

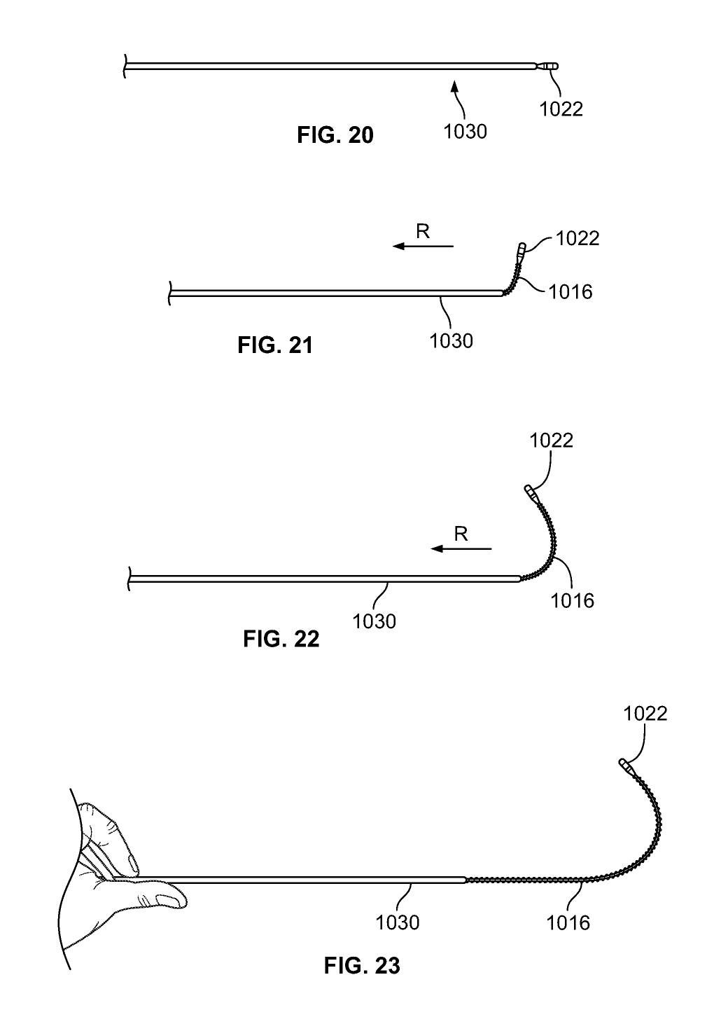

FIGS. 20-23 show a distal treatment section 1016 of a cryoablation catheter being deployed. With reference to FIG. 20, an outer sheath or sleeve 1030 is shown. It surrounds a plurality of tube members 1016. The tubes are made of a shape memory alloy in this embodiment. The outer sheath 1030 holds or constrains the transport tubes, preventing the transport tubes from assuming a preset shape. The outer sheath is desirable flexible enough to be navigated through the vasculature, or through a guide catheter already positioned in the vasculature, but rigid enough to retrain the shape member tubes in an undeployed configuration. Exemplary materials for the outer sheath or sleeve include polymers such as, the polymers and materials used in endovascular applications. Non-limiting examples include polyethylene (PE), polypropylene (PP), polyvinyl chloride (PVC) and fluorocarbons (PTFE).

Upon reaching the destination or target tissue (not shown), the sheath 1030 and treatment section 1016 are moved relative to one another such that the distal treatment section projects from the end of the sheath. For example, the sheath may be retracted (R) by manipulating the sheath by hand at the proximal end of the catheter, or more sophisticated structures may be incorporated such as thumb pad or lever as described in U.S. Pat. No. 6,984,230 to Scheller et al.

With reference to FIG. 21, the tip 1022 is shown immediately curving as it extends from the sheath to an offset position. A diagnostic or imaging modality may be employed such as fluoroscopy to confirm location and deployment of the distal treatment section. Radio-opaque bands or markers may be carried on the distal treatment section 1016 (not shown) to facilitate location and visualization of the device in situ.

FIG. 22 shows distal treatment section 1016 being further deployed from sheath 1030. Treatment section 1016 continues to assume its preset shape.

FIG. 23 shows distal treatment section 1016 fully deployed. The curved configuration shown in FIG. 23 is, for example, a predetermined deflection to match an anatomy of a target tissue. Exemplary tissues and targets to be treated include myocardial tissue including without limitation the myocardial tissue of the left or right atrium. However, the shape of the curve or deflection in the second configuration may vary widely and the physician may manipulate the shape by controlling the degree of deployment, or selecting a different preset shape to match a particular anatomy or target area.

In embodiments a cryoablation method comprises providing a cryoablation catheter including a distal treatment section. The distal treatment section is positioned in the vicinity of the target tissue. The distal treatment section is partially deployed, namely, the sheath is retracted, allowing the distal treatment section to partially deflect into its preset shape. The location of the tip and distal treatment section are confirmed to be in proper position relative to the anatomy and target tissue to be ablated.

Upon confirmation of the location of the distal treatment section, it is further deployed or released until the distal treatment section is fully deployed and in proper position relative to the target tissue. Preferably the treatment section or freeze zone is contacting the segment of tissue to be ablated. Optionally, the position is reconfirmed. Then, the catheter is activated to cause the treatment section to stick to the tissue, locking its position in place. Cooling power is continued until the target tissue/lesion has been sufficiently ablated. For example, as discussed further herein, in the case of treating atrial fibrillation, a full thickness or transmural linear lesion may be effected. The cooling power is then halted to allow the distal treatment section to thaw, and de-stick from the tissue. The distal treatment section may then be retracted within the outer sheath, and the catheter removed from the target area. In embodiments a controller measures temperature, flow rate, and time elapsed, and halts the cooling power once a threshold condition is reached. In embodiments, the cooling power is halted after a time period has elapsed.

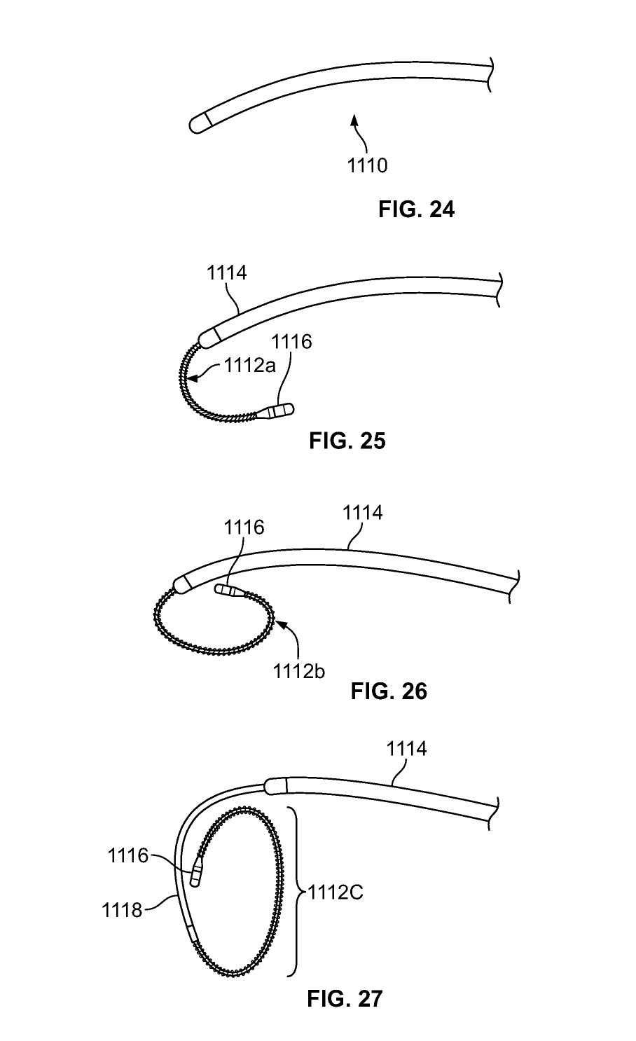

FIGS. 24-27 show another cryoablation catheter similar to that described in connection with FIGS. 20-23 except the distal treatment section includes a plurality of preformed (or preset) treatment shapes. More specifically, FIG. 25 shows distal treatment section having a concave portion 1112a. FIG. 26 shows distal treatment section having a convex portion 1112b. FIG. 27 shows distal treatment section having a flat portion 1112c. The distal treatment section assumes one of the predetermined shapes based on the travel distance the tip 1116 is ejected from the outer sheath 1114.

The shape of the distal treatment section is thus conveniently changed by adjusting the travel distance that the tip is ejected from the outer sheath. In this embodiment, the distal treatment section utilizes the property of elasticity so that it may automatically return to (or assume) its pre-formed shape. This embodiment of the invention avoids plastic deformation and operates using a different principle than malleable elongate shafts which do not spring back to an original shape when unconstrained. As will be described in more detail herein, the shapes can be preset to treat a plurality of different anatomical regions.

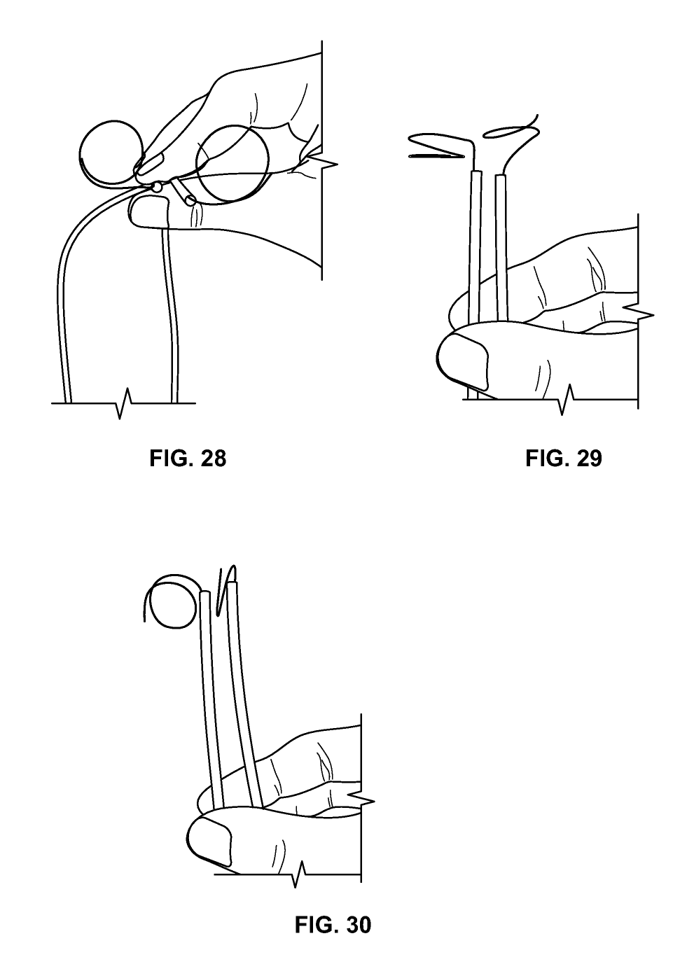

The preformed treatment shapes may have a wide variety of geometries. FIGS. 28-29, for example, show circular loops formed perpendicular to the axis of the sheath. FIG. 30 shows a circular loop formed substantially in the same plane as the axis of the sheath. Circular shapes may serve to treat circular-shaped anatomical regions such as the entries to the pulmonary vessels in connection with trying to eliminate atrial fibrillation.

Additionally, the plurality of different treatment shapes may have 1D, 2D or 3D configurations. One treatment shape may lie in the same plane as another treatment shape, i.e., coplanar. Alternatively, one treatment shape may lie outside the plane of another treatment shape.

The size of the preformed treatment shapes may vary. In embodiments the size and shape of the treatment section matches the anatomical surfaces in the heart. The size may be adjusted to suit different individuals.

The number of preformed treatment shapes per instrument may vary and be determined based on the target tissue or application. As described further herein, a treatment section having 1, 2 or 3 preset shapes may be desirable. However, a treatment section may be designed having 2-10 shapes, or perhaps 3-5 shapes, and in some embodiments only 3 preset shapes.

In other embodiments, a preformed stylus or preformed outer shell layer may be incorporated in the distal treatment section to create the above described preset shapes.

The preformed members may be shape set by wrapping or otherwise manipulating the member around a mandrel or mold. The entire fixture (mold and member) is then submerged in a temperature controlled bath for a sufficient time period to set the shape. In embodiments, the members exhibit superelastic properties. Examples of suitable shape set materials include without limitation Nitinol.

In yet another embodiment, a pull wire and optional spine element may be incorporated into the distal treatment section to articulate and deflect the treatment section to the desired curvature.

Examples of various components described herein are shown and described in the following patent applications each of which is incorporated by reference in its entirety: International Patent Application No. PCT/US2014/56839, filed Sep. 22, 2014; International Patent Application No. PCT/US2014/59684, filed Oct. 8, 2014; and international Patent Application No. PCT/US2015/024778, filed Apr. 7, 2015.

Elliptical Shaped Distal End Section

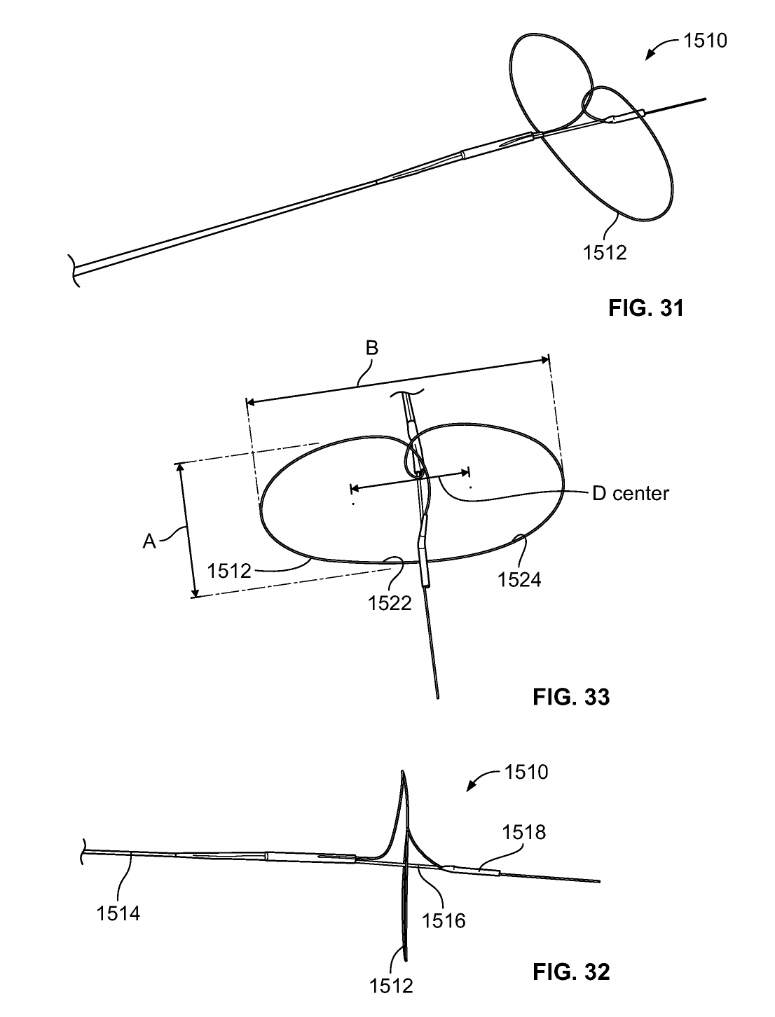

FIGS. 31-33 are various views showing the distal section of a cryoablation catheter 1510 having an elliptical-shaped energy deliver member 1512 when deployed.

With reference to FIG. 32, the deployed configuration is substantially planar. The defined plane is perpendicular to the shaft 1514 of the catheter.

An elongate control member 1516 is shown extending from the catheter shaft 1514 to a distal tip 1518 where the energy deliver member 1512 is affixed to the control member. As will be discussed further herein, manipulation of the control member 1516 can serve to adjust deployment and shape of the energy delivery element.

With reference to FIG. 33, the elliptical shape is shown being formed of two overlapping circles 1522, 1524 separated by a distance (D_{center}). The shape comprises a minor axis (A) and a major axis (B). In embodiments the major axis and minor axis range from 2-6 cm, and 1-2 cm, respectively.

In embodiments, and as will be discussed further herein, the distance (D_{center}) may be adjusted by manipulation of the control member. The circles may be spread further apart, affecting the eccentricity of the elliptical shape.

In another embodiment, the major axis is biased to elastically deform prior to the minor axis as the catheter is deployed against tissue. Although the shape of the deployed distal section is shown as being substantially elliptical, adjustments to the shape may be made to provide a different shape. Exemplary shapes include, without limitation, oval, heart, egg, butterfly, ellipse, FIG. 8, and clover. Still the invention may include other shapes except where specifically excluded in the appended claims.

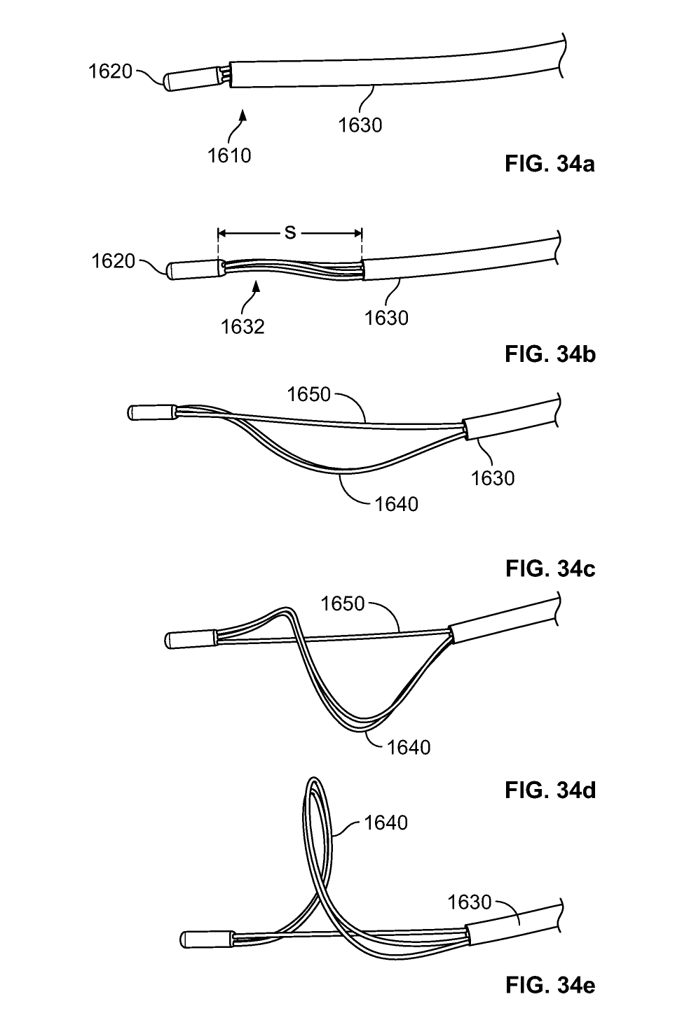

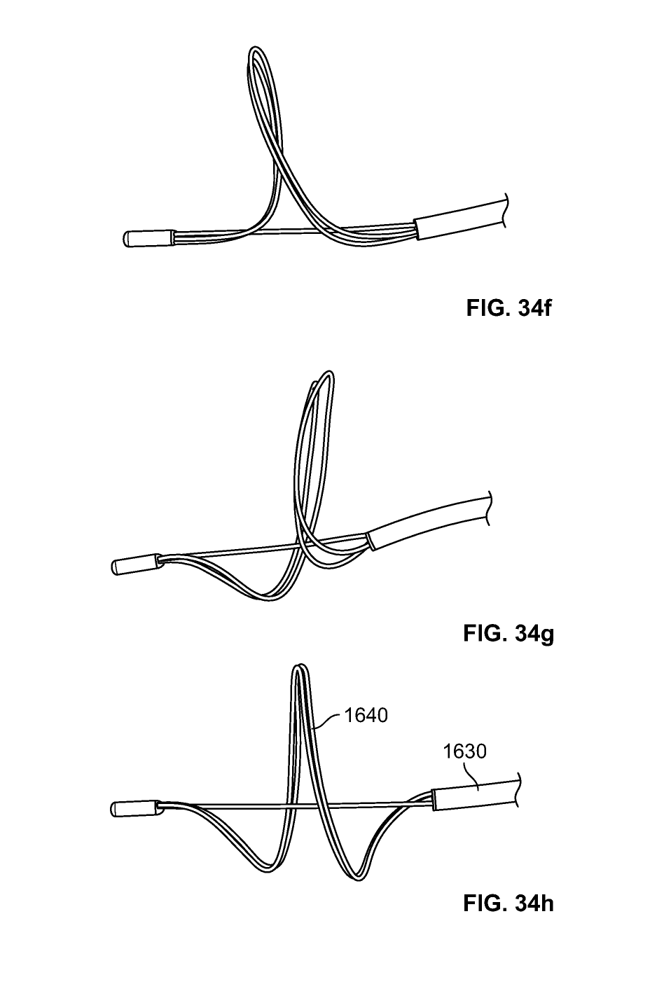

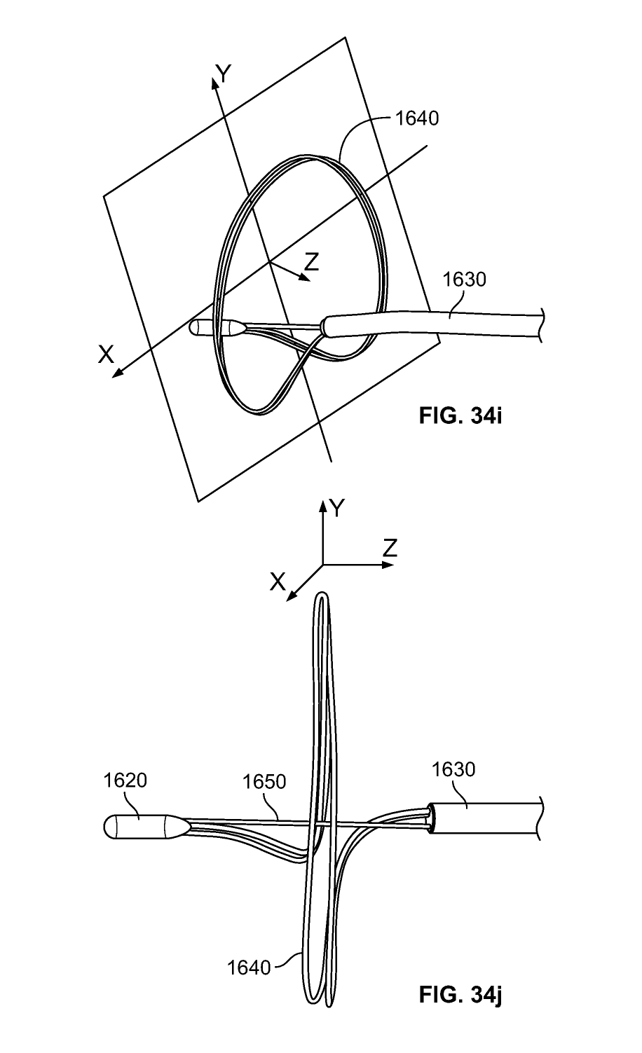

FIGS. 34a-34j are illustrations sequentially showing a distal end section of a cryoablation catheter 1610 being deployed from a first low profile (e.g., an elongate and substantially linear) configuration into a second large profile (e.g., an ovular and substantially planar) configuration.

FIG. 34a is an initial undeployed configuration of the distal end section 1610 of the catheter. A distal tip 1620 is shown adjacent (or protruding) from an elongate shaft 1630.

FIG. 34b shows the distal tip spaced (S) from the elongate shaft. This step is carried out by moving the shaft relative to the tip or vice versa. The components may be moved by manual manipulation, or as discussed further herein, the components may be controlled using other mechanisms such as, for example, actuating features on a handle. A non-limiting exemplary length of space (S) is between 0 and 2, or more preferably between 0.5 and 1 cm.

FIG. 34b also shows a bundle 1632 extending from the catheter shaft 1630 to the distal tip 1620. The bundle 1632 comprises an energy delivery member 1640 and control member 1650 to manipulate the shape of the energy delivery member 1640 once ejected from the catheter shaft 1630 as will be discussed further herein.

FIGS. 34c-34h show intermediate deployment of the energy delivery member 1640. In particular, the energy delivery member commences in a slightly curved shape corresponding to that shown in FIG. 34c, and progresses to a three dimensional shape (e.g., spiral, coil) corresponding to that shown in FIG. 34h. The shape of the energy delivery member 1640 is assumed as it is ejected from the catheter shaft 1630.

FIGS. 34i-34j show adjustment of the three dimensional shape shown in FIG. 34h from a 3D shape to a 2D shape. As shown in FIG. 34i, for example, the shape of the energy delivery member 1640 is now ovular and substantially confined to the XY plane. This step may be carried out by further ejecting the energy delivery element 1640 and manipulation of the control member relative to the sheath. As the energy delivery member 1640 is released, it assumes a pre-set shape. Additionally, because the control member is connected to the distal tip 1620, and the end of the energy delivery member 1640 is also connected to the distal tip, manipulation of the control member 1650 adjusts the pre-set shape of the energy delivery element, to a desired shape that makes greater contact with the target anatomy. The eccentricity and size may be adjusted.

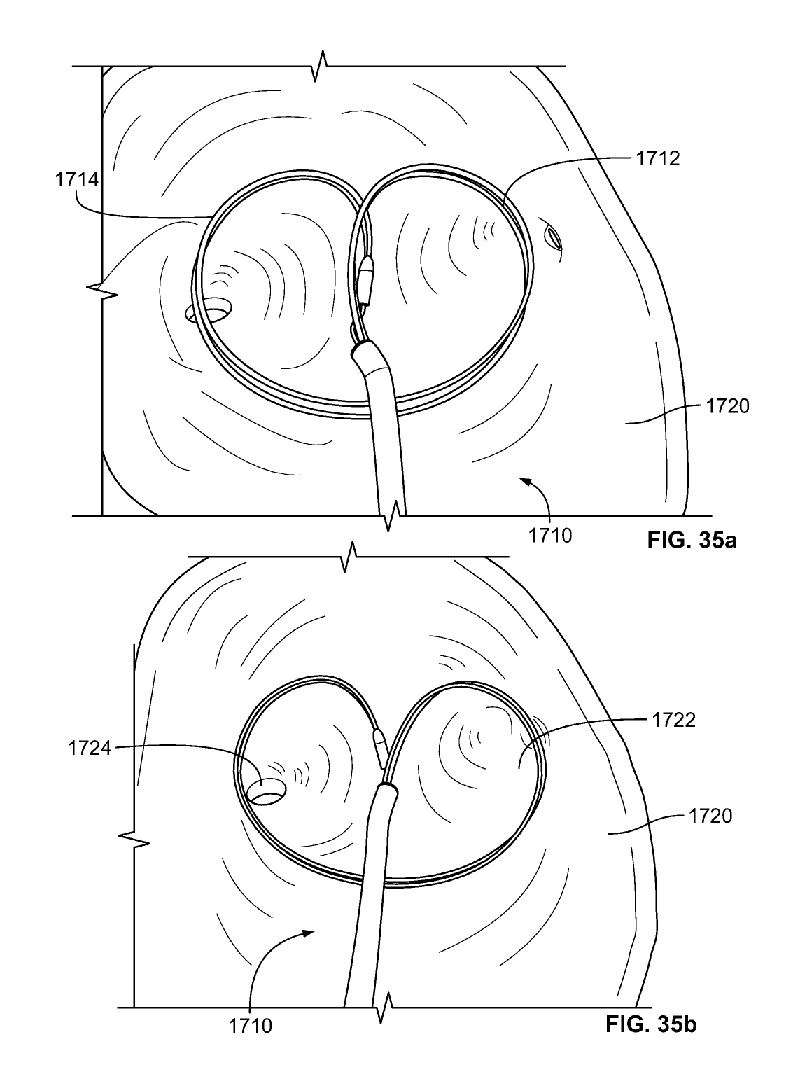

FIGS. 35a-35b illustrate adjusting the shape of the deployed distal end section 1710 in a 3D printed model of the left atrium 1720. In particular, and with reference first to FIG. 35a, loops 1712, 1714 are positioned on the inside or across the vein entries 1722, 1724. Then, after the distal end section has been adjusted as described herein and shown in the FIG. 35b, the loops 1712, 1714 circumscribe vein entries 1722, 1724. The increased eccentricity of the deployed distal treatment section enables formation of a more complete lesion that engulfs both the pulmonary vein entries 1722,1724.

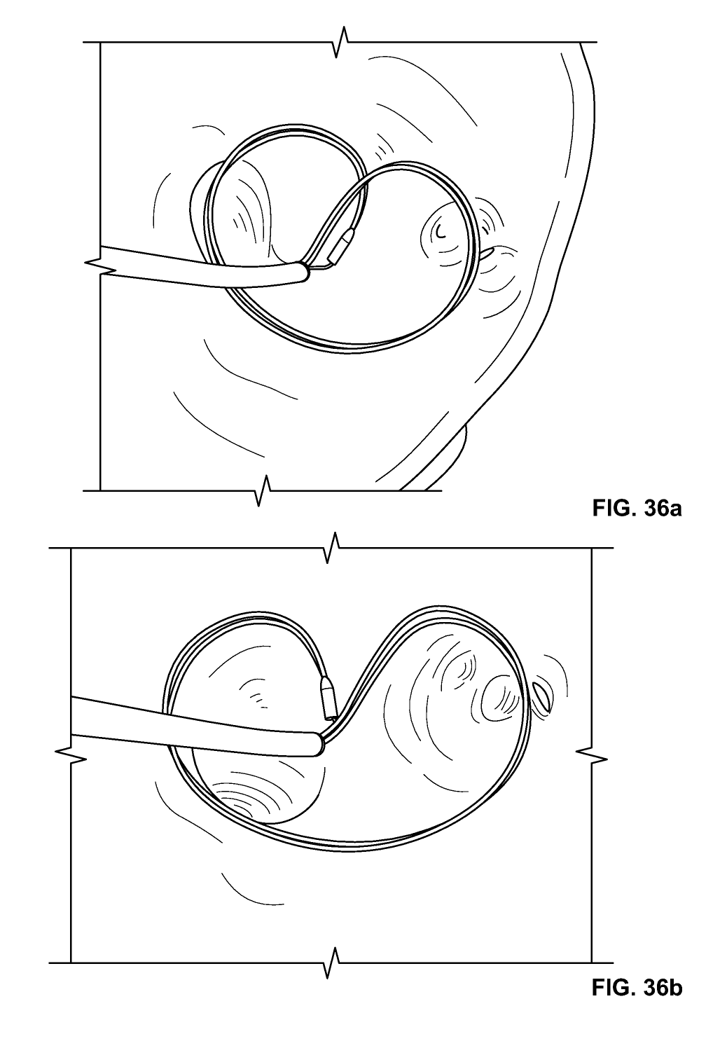

FIGS. 36a-36b also illustrate the step of adjusting the distal end section in an artificial tissue model. Similar to FIGS. 35a-35b, after the energy delivery element is ejected and assumes a first shape corresponding to that shown in FIG. 36a, the eccentricity of the closed curve is increased to capture the vein entries as shown in FIG. 36b.

As mentioned above, the energy delivery elements may be made of a wide variety of materials. In embodiments, the energy delivery elements are made of a shape memory material having a pre-set shape as illustrated herein. In embodiments, the shape takes a clover or heart shape which has a biased deformation along its major axis. In a sense, the leafs of the 2D clover rotate inward towards one another, causing the overall width of the clover to be affected prior to the height.

In embodiments, the energy delivery elements have an elasticity similar to the target tissue so as not to score, puncture, or otherwise damage the tissue as the device is moved and deployed into position. In embodiments, the elasticity of the energy delivery elements is not greater than the elasticity of the tissue, and in other embodiments, the elasticity is not substantially less than that of the tissue (e.g., the endocardial wall of the heart). Consequently, the energy delivery element is able to hold its shape and be firmly pressed against the endocardium. The surgeon may deploy the energy delivery members into close proximity with the target endocardium surfaces without collateral damage, and then adjust the position, angle, eccentricity and shape of the deployed element into final position.

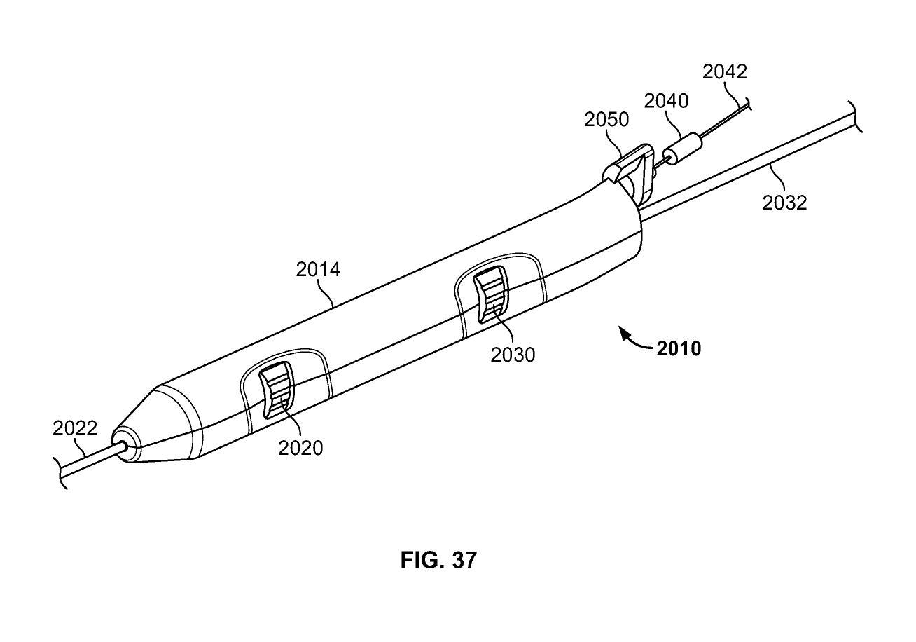

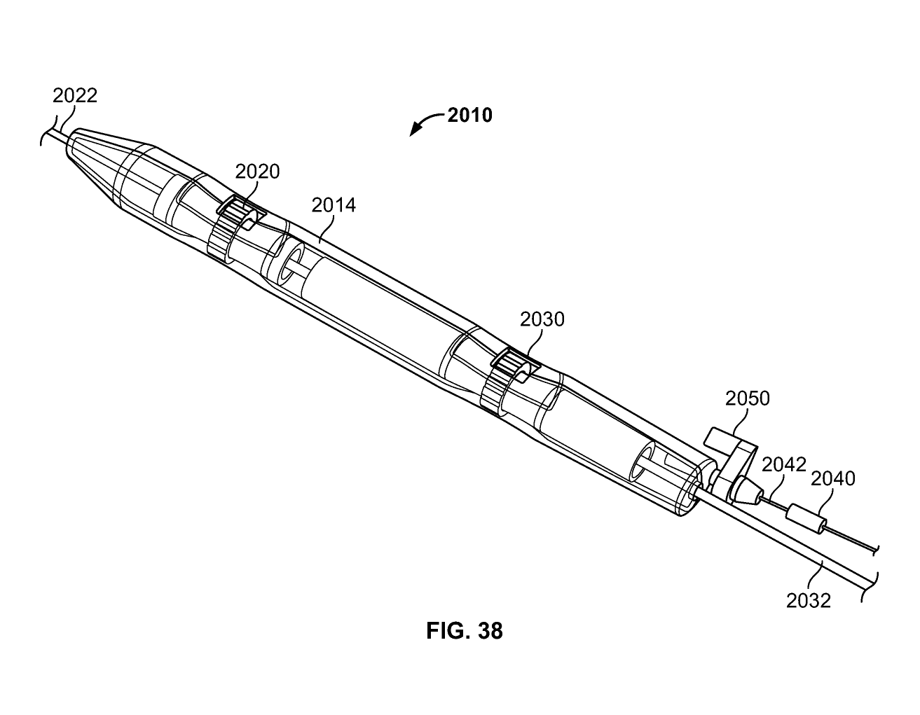

FIGS. 37-38 show a handle 2010 having a plurality of actuating members 2020, 2030, 2040, 2050 to deploy the energy delivery elements as described herein. First wheel 2020 is shown rotatably sitting in the handle body 2014. First wheel is in threaded engagement with the outer sheath for moving (namely, retracting or advancing) the outer sheath 2022 relative to the distal tip of the device. As described above, this step exposes or provides space between the distal tip and the end of the sheath so that the energy delivery element may be ejected from the sheath and assume its pre-set shape.

Second wheel 2030 is shown rotatably sitting in the handle body 2014. Second wheel is in threaded engagement with a driver tube 2032. The driver tube is connected with (e.g., coaxially surrounds) the energy delivering members (not shown) to eject/withdraw the energy delivery tubes from the sheath when the second wheel is rotated.

Grip 2040 is shown fastened to control wire 2042. Grip 2040 may be rotated or moved axially to move the distal tip of the catheter relative to the shaft. As described herein, rotating the control member serves to adjust the eccentricity or shape of the deployed energy delivery member to fit the target tissue. Once in a desired position and shape, the surgeon may lock the control member using lever 2050. Lever 2050 may be linked or include a cam member or gear to clamp and hold control wire 2042 in place when the lever is rotated.

In the embodiment shown in FIGS. 37-38, the actuators are arranged from the distal end to the proximal end in the order to be actuated corresponding to deploying the catheter working end into its target arrangement. It should be understood however that a wide a range of actuators mechanisms and features may be included in a handle and are intended to be included within the present invention except where excluded by the appended claims.

Applications

An exemplary application is endovascular based cardiac ablation to create elongate continuous lesions. As described herein, creating elongate continuous lesions in certain locations of the heart can serve to treat various conditions such as, for example, atrial fibrillation or atrial flutter.

The Cox maze procedure to treat atrial fibrillation has been performed using radio frequency ablation catheters in both transthoracic epicardial approaches and transvascular endocardial approaches.

In transthoracic epicardial approaches, catheters or small probes are used to create linear lesions in the heart wall along lines corresponding to the maze of the Cox maze procedure. In the transvascular endocardial approaches, a catheter is navigated through the vasculature of the patient to the atrium, pressed against the inner wall of the atrium, and energized to create lesions corresponding to the maze of the Cox maze procedure.

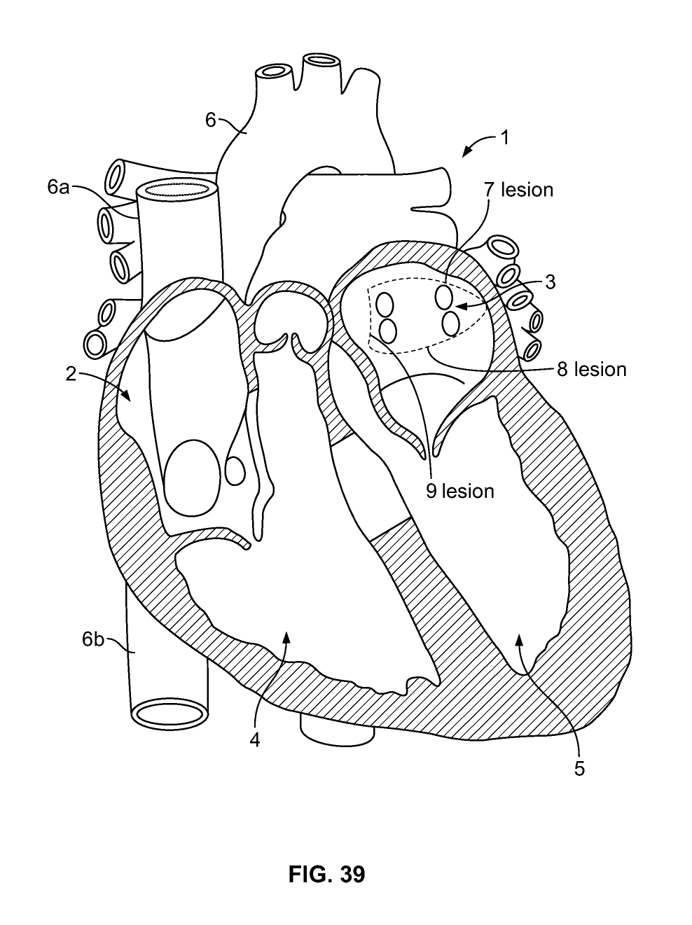

FIG. 39 shows examples of target sections of tissue and lesions in a Cox Maze procedure. Basic structures of the heart include the right atrium 2, the left atrium 3, the right ventricle 4 and the left ventricle 5. Catheters may be inserted into these chambers of the heart through various vessels, including the aorta 6 (accessed through the femoral artery), the superior vena cava 6a (accessed through the subclavian veins) and the inferior vena cava 6b (accessed through the femoral vein).

The following discussion will focus on embodiments for performing the left atrium lesion of the Cox maze VII procedure, but the procedure for producing these lesions can be used to create other lesions in an around the heart and other organs. Additional lesions of the Cox maze VII procedure, as well as other variations of the Cox Maze treatments may be carried out using steps and devices described herein. Additional techniques and devices are described in international patent application nos. PCT/US2012/047484 to Cox et al. and PCT/US2012/047487 to Cox et al. corresponding to International Publication Nos. WO 2013/013098 and WO 2013/013099 respectively.

In FIG. 39, a few of the left atrium lesions of the Cox maze VII lesion are illustrated. Cox maze lesions 7, 8 and 9 are shown on the inner wall of the left atrium. These correspond to the superior left atrial lesion (item 7) spanning the atrium over the left and right superior pulmonary vein entries into the atrium, the inferior left atrial lesion (item 8) spanning the atrium under the left and right inferior pulmonary vein entries into the atrium, and the vertical lesion (item 9) connecting the superior left atrial lesion and inferior left atrial lesion so that the right pulmonary veins are within the area defined by the lesions.

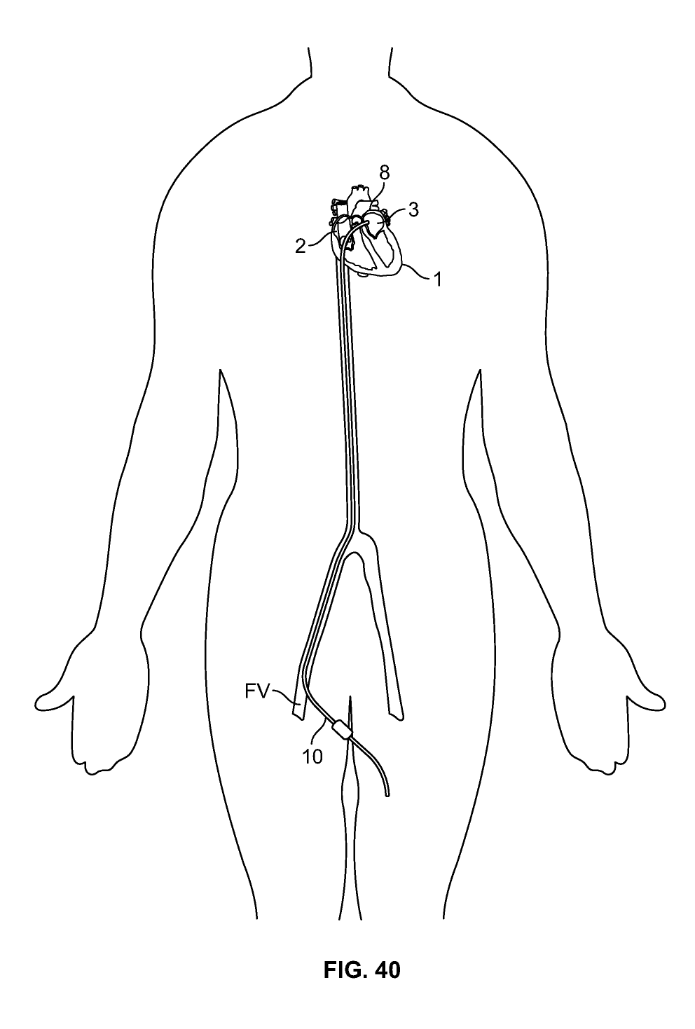

FIG. 40 illustrates one technique to reach the left atrium with the distal treatment section of a catheter. A peripheral vein (such as the femoral vein FV) is punctured with a needle. The puncture wound is dilated with a dilator to a size sufficient to accommodate an introducer sheath, and an introducer sheath with at least one hemostatic valve is seated within the dilated puncture wound while maintaining relative hemostasis. With the introducer sheath in place, the guiding catheter 10 or sheath is introduced through the hemostatic valve of the introducer sheath and is advanced along the peripheral vein, into the target heart region (e.g., the vena cavae, and into the right atrium 2). Fluoroscopic imaging can be used to guide the catheter to the selected site.

Once in the right atrium 2, the distal tip of the guiding catheter is positioned against the fossa ovalis in the intraatrial septal wall. A needle or trocar is then advanced distally through the guide catheter until it punctures the fossa ovalis. A separate dilator may also be advanced with the needle through the fossa ovalis to prepare an access port through the septum for seating the guiding catheter. The guiding catheter thereafter replaces the needle across the septum and is seated in the left atrium through the fossa ovalis, thereby providing access for devices through its own inner lumen and into the left atrium.

Other left atrial access methods may be suitable substitutes for using the ablation device assembly of the present invention. In one alternative, a “retrograde” approach may be used, wherein the guiding catheter is advanced into the left atrium from the arterial system. In this variation, the Seldinger technique may be employed to gain vascular access into the arterial system, rather than the venous, for example, at a femoral artery. The guiding catheter is advanced retrogradedly through the aorta, around the aortic arch, into the ventricle, and then into the left atrium through the mitral valve.

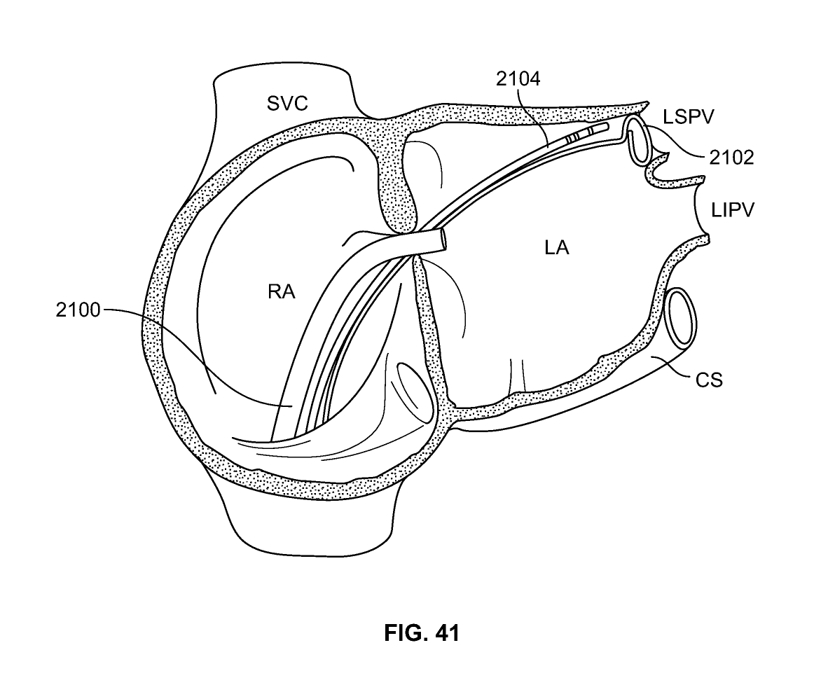

FIGS. 41-45 illustrate a method for deploying an elliptical shaped catheter in the left atrium and around pulmonary vein entries for treating various heart conditions such as atrial fibrillation.

With reference first to FIG. 41, a cross sectional view of the heart includes the right atrium RA, left atrium LA, left superior pulmonary vein LSPV entry, and left inferior pulmonary vein LIPV entry. Guide catheter 2100 is shown extending through the septum and into the left atrium as described above. Mapping catheters 2102, 2104 are shown positioned in the left atrium for monitoring electrical signals of the heart. Examples of mapping catheters include the WEBSTER® CS Bi-Directional Catheter and the LASSO® Catheter, both of which are manufactured by Biosense Webster Inc. (Diamond Bar, Calif. 91765, USA).

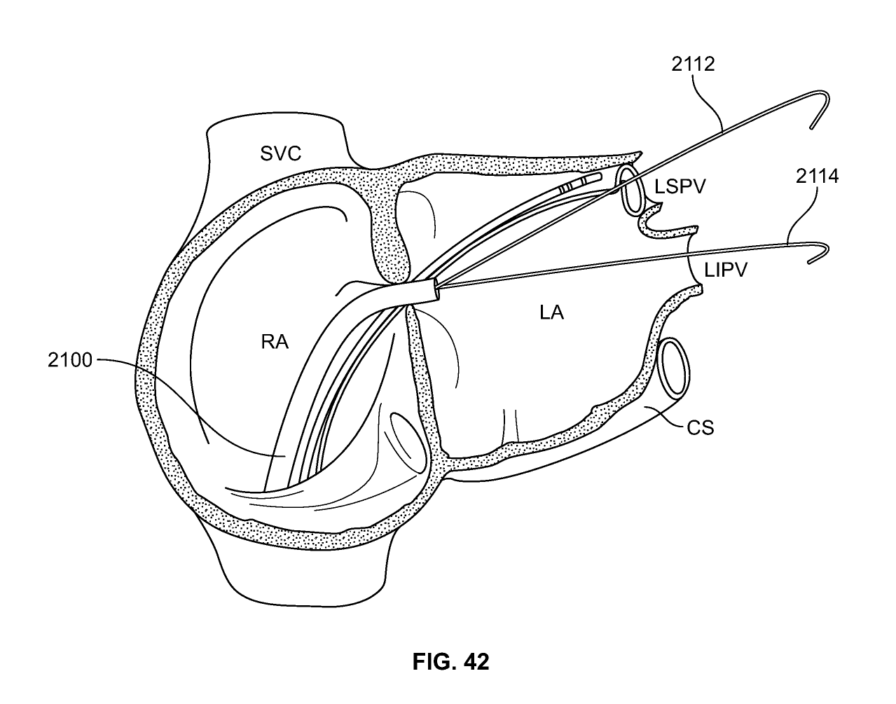

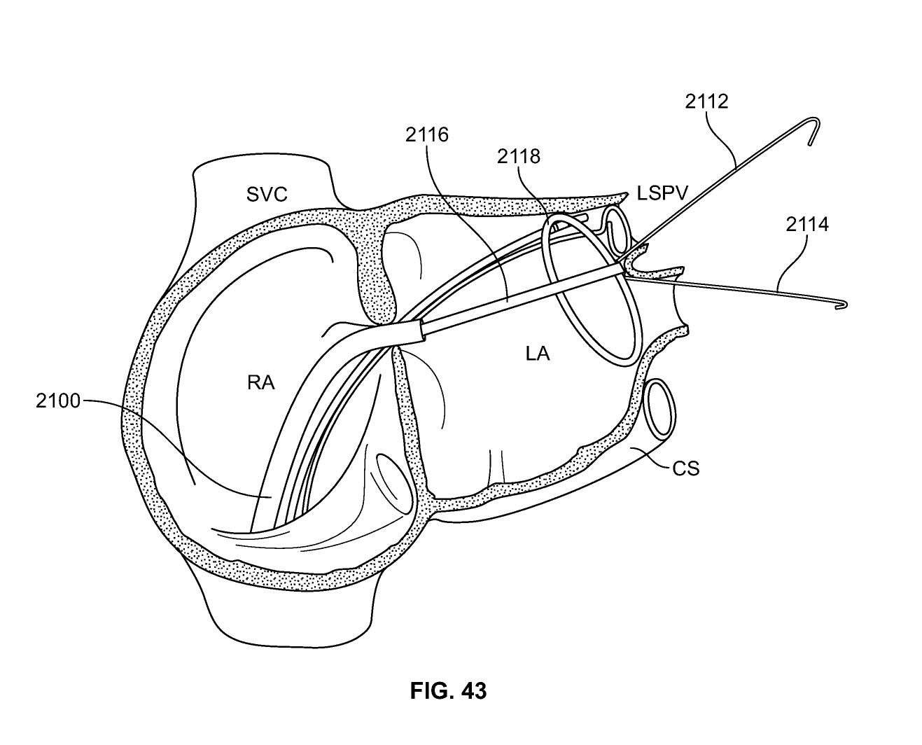

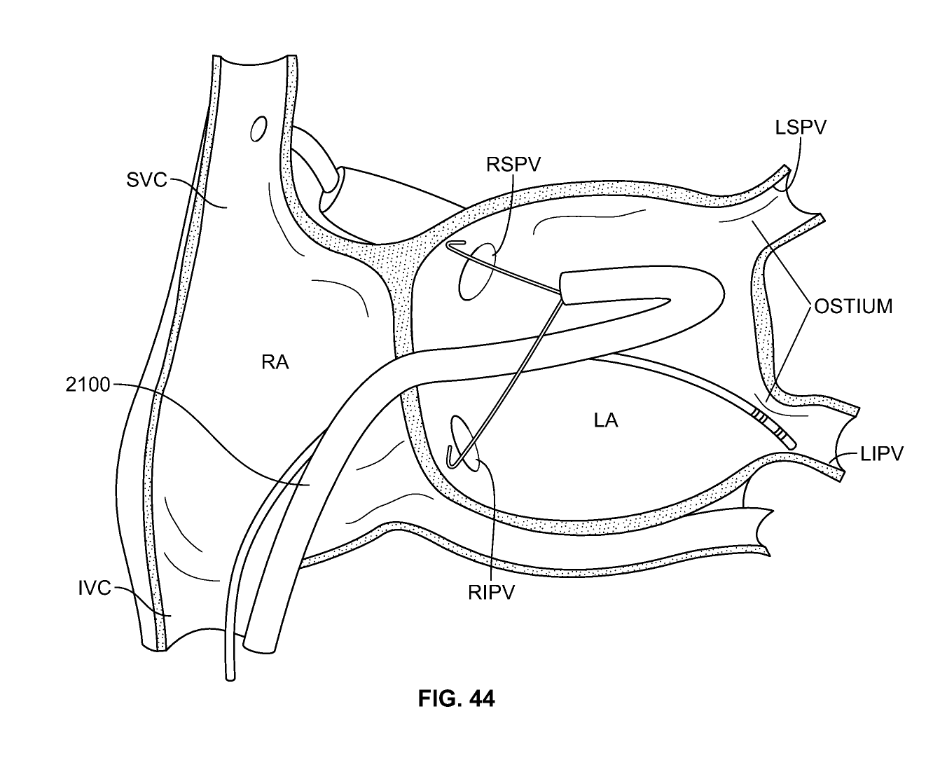

FIG. 42 shows placement of guidewires 2112, 2114 into the LSPV and LIPV respectively.

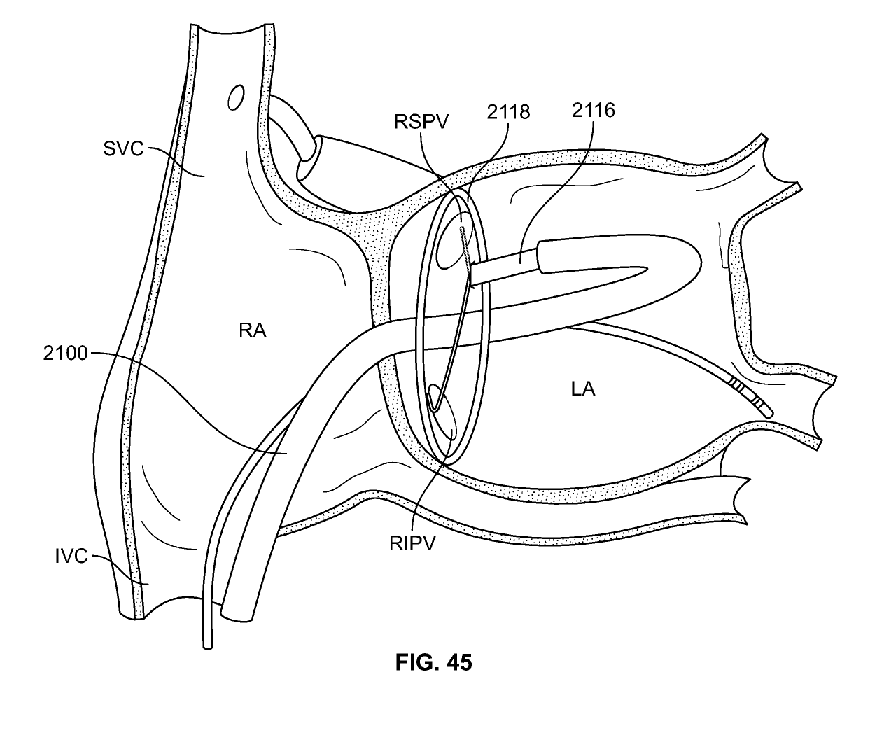

FIG. 43 illustrates a distal section of the cryoablation catheter 2116 advanced through the guide sheath and over the guidewires 2112, 2114 to centrally align between the left pulmonary vein entries. The energy element 2118 is shown having a circular shape and urged against the endocardium. As described herein the shape may be adjusted to better make contact with the tissue, and to form an ovular continuous lesion which engulfs or surrounds the PV entries. In embodiments the shape is modified such that the eccentricity (E) is adjusted from E=0 (corresponding to a circular shape) to 1 (corresponding to a substantially elliptical shape).

FIGS. 44-45 illustrate formation of a ring shaped lesion around the right pulmonary veins. In contrast to the somewhat linear positioning of guide sheath shown in FIGS. 41-43, the guide sheath 2100 in FIG. 44 is deflected nearly 180 degrees to aim towards the right pulmonary vein entries. In embodiments, guidewires are advanced from the guide sheath and into the right superior and inferior pulmonary veins. The cryoablation catheter 2116 is advanced over the wires and in a position between the two right pulmonary vein entries. FIG. 45 shows the energy element 2118 in a circular shape and snugly pressed to the endocardium. As described herein the shape may be adjusted to better make contact with the tissue, and to form an elongate ring shaped continuous lesion which engulfs or surrounds the PV entries.

In embodiments, the device and method is adapted and intended to create a number of lesions including ring or elliptical shaped lesions which engulf or circumscribe one or more pulmonary vein entries in the left atrium (e.g., to surround both left superior and inferior pulmonary vein entries, or both right pulmonary superior and inferior vein entries). In other embodiments, an elongate linear tip is provided to make continuous, more linear, lesions that span the atrium over the left and right superior pulmonary vein entries into the atrium, under the left and right inferior pulmonary vein entries into the atrium and/or a vertical lesion on the right of the right superior and inferior vein entries into the atrium. The lesions are preferably continuous, not a series of spots such as in some prior art point-ablation techniques. In accordance with the designs described above, the cryoenergy and heat transfer is focused on the endocardium, and intended to create the lesion completely through the endocardium.

Preferably, in embodiments, the catheters achieve cooling power without vapor lock by transporting the cooling fluid near its critical point in the phase diagram. The distal treatment section designs described herein create elongate continuous lesions spanning the full thickness of the heart wall. The heat sink associated with the warm blood flow through the chambers of the heart is mitigated or avoided altogether because the ablation catheter is positioned within the heart chamber and directs the treating energy from the endocardium to the pericardium, or from the inside out.

Multiple endovascular products are described herein having a number of advantages including, for example: a) maintaining pressures of near-critical nitrogen below the maximum tolerance of −600 psi for endovascular catheter material, b) containing leaks to eliminate the dangers arising there from, and c) controllably deploying distal treatment sections to treat a plurality of tissue areas having different curvatures. A cardiac ablation catheter in accordance with the principals of the present invention can be placed in direct contact along the internal lining of the left or right atrium, thereby avoiding most of the massive heat-sink of flowing blood inside the heart as the ablation proceeds outward.

In addition to that described above, the devices described herein may have a wide variety of applications including, for example, endoscopic cryotherapy. Candidate tumors to be ablated with cryoenergy include target tissues and tumors in the bronchial tree or lung as well as tissues in the upper and lower GI. The devices described herein may also be applied to destroy or limit target tissues in the head and neck.

Many modifications and variations of the present invention are possible in light of the above teachings. It is, therefore, to be understood that within the scope of the appended claims, the invention may be practiced otherwise than as specifically described.

We claim:

1. A cryoablation catheter for creating at least one lesion in tissue, the catheter comprising:
an elongate shaft comprising an intermediate section and a distal tip movable relative to the intermediate section;
at least one elongate control member extending along the intermediate section and secured to the distal tip, the elongate control member being movable relative to the intermediate section for causing movement of the distal tip relative to the intermediate section; and
at least one energy delivery member extending along the intermediate section to the distal tip, the at least one energy delivery member comprising a linear first configuration and an elliptical second configuration,
wherein manipulation of the control member adjusts a shape of the at least one energy delivery member.

2. The catheter of claim 1, wherein the distal tip is axially and rotationally movable relative to the intermediate section.

3. The catheter of claim 1, wherein the at least one energy delivery member comprises a fluid inflow tube to deliver a cryogen to the distal tip.

4. The catheter of claim 1, wherein the at least one energy delivery member comprises a fluid return tube to transport a cryogen away from the distal tip.

5. The catheter of claim 1, wherein the control member is a fluid return tube for transporting a cryogen away from the distal tip.

6. The catheter of claim 1 wherein the second elliptical configuration comprises a first circular portion and second circular portion overlapping with the first circular portion.

7. The catheter of claim 6, wherein a center of the first circular portion is separated from a center of the second circular portion by a distance D.

8. The catheter of claim 1, wherein the control member and the energy delivery member form a telescoping arrangement.

9. The catheter of claim 1, wherein the second configuration is formed by a single continuous tubular element.

10. The catheter of claim 1, further comprising an outer sheath comprising a proximal end and a distal end, the outer sheath and elongate shaft being axially slideable relative to one another.

11. The catheter of claim 1, further comprising an insulating layer surrounding at least a portion of the elongate shaft.

12. The catheter of claim 1, wherein the at least one energy delivery member comprises a superelastic material.

13. The catheter of claim 1, wherein the elliptical second configuration has a shape adapted to create a continuous lesion in a heart encompassing both LSPV and LIPV entries.

14. The catheter of claim 1, further comprising a stylus element extending along the at least one energy delivery member, wherein the stylus element is spring biased.

15. The catheter of claim 1, wherein the at least one energy delivery member further comprises a three dimensional intermediate configuration occurring between the linear first configuration and the elliptical second configuration.

16. The catheter of claim, wherein the elliptical second configuration is automatically assumed when the at least one energy delivery member is not surrounded by an outer sheath.

17. The catheter of claim 1, wherein the at least one energy delivery member is spring biased.

18. The catheter of claim 1 wherein the control member is a single wire extending from the intermediate section to the distal tip.

19. An endovascular cryoablation catheter for creating at least one lesion in target tissue, the catheter comprising:
an elongate shaft having an intermediate section, a distal treatment section and at least one energy delivery member extending there through, wherein (i) the distal treatment section comprises a low-profile undeployed configuration and a high-profile substantially planar deployed configuration, and (ii) the deployed configuration comprises a first closed curve having a first center and a second closed curve having a second center, and
a means to control movement of the first closed curve relative to the second closed curve such that a distance between the first center and the second center can be adjusted.

20. The catheter of claim 19, wherein a flow of near critical fluid through the at least one energy delivery member is used to transfer heat from the target tissue to the distal treatment section of the catheter thereby creating the at least one lesion in the tissue.

21. The catheter of claim 20, wherein the lesion is continuous.

22. The catheter of claim 19, wherein the means to control movement of the first closed curve relative to the second closed curve is an elongate control member extending along the intermediate section and secured at the distal treatment section.

23. An endovascular cryoablation catheter for creating at least one continuous lesion in target tissue, the catheter comprising:
an elongate shaft comprising an intermediate section and a distal treatment section having at least one tubular energy delivery member extending there through,
wherein the distal treatment section comprises a low-profile undeployed configuration and a high-profile substantially planar deployed configuration, and
wherein the deployed configuration comprises a first leaf and a second leaf in telescoping and rotatable cooperation with the first leaf such that the first leaf and second leaf may be moved between a substantially concentric arrangement and an eccentric arrangement.

24. The catheter of claim 23, further comprising a flow of near critical fluid through the at least one tubular energy delivery member to transfer heat from the target tissue to the distal treatment section of the catheter thereby creating the at least one continuous lesion in the tissue.

25. A method of creating a continuous lesion in cardiac tissue in a heart, the method comprising:
inserting a catheter comprising an inner elongate shaft having a distal treatment section, at least one cryogen delivery tube and an outer sheath axially movable relative to the inner elongate shaft, into a patient's vasculature;
navigating the distal treatment section of the catheter to the heart and through an opening in the heart until the distal treatment section is within a space in the heart;
exposing the distal treatment section of the elongate shaft by moving the outer sheath relative to the distal treatment section;
transforming the distal treatment section from a linear low profile first shape, to an intermediate shape, to a planar curved second shape, wherein the step of transforming comprises adjusting the eccentricity of the intermediate shape into the curved second shape;
contacting the curved second shape with the cardiac tissue; and
circulating a near critical fluid through the at least one cryogen delivery tube while the distal treatment section is in contact with the cardiac tissue.

26. The method of claim 25, wherein the transforming step is performed by rotating a pair of circles away from one another until the curved second shape is formed, and wherein the curved second shape is selected from the group consisting of a heart, oval, egg, clover, butterfly, and a FIG. 8.

27. The method of claim 26, wherein the space in the heart is the left atrium and the method further comprises advancing a guide sheath through a septum and into the left atrium thereby providing access to the cardiac tissue.

28. The method of claim 27, further comprising advancing a first guidewire through the guide sheath and into a first PV entry.

29. The method of claim 28, further comprising advancing a second guidewire through the guide sheath and into a second PV entry.

30. The method of claim 29, further comprising advancing the catheter simultaneously along the first and second guidewires towards the first and second PV entries, thereby centering the distal treatment section of the catheter between the first and second PV entries.

31. The method of claim 30, wherein the first and second PV entries are the LSPV and LIPV entries respectively.

32. The method of claim 31, further comprising creating at least one single continuous oval-shaped lesion along the cardiac tissue encircling both the LSPV and the LIPV entries.

33. The method of claim 29, further comprising advancing a pacing catheter for monitoring electrical activity of the heart.

34. The method of claim 25, wherein the transforming step is performed by manipulating a control member.

35. The method of claim 34, wherein manipulating the control member comprises rotational motion.

36. The method of claim 25, further comprising halting the circulating step when a threshold condition is met, wherein the threshold condition is one condition selected from the group consisting of: length of lesion, thickness of lesion, time elapsed, energy transferred, temperature change, pressure change, flowrate change, and power change.

37. The method of claim 36, wherein the halting step is based on time elapsed.

38. The method of claim 37, wherein the time elapsed is at least 2 minutes.

39. The method of claim 36, further comprising a thawing step, allowing the cardiac tissue to thaw.

40. The method of claim 39, further comprising repeating the circulating step while the distal treatment section remains in contact with the cardiac tissue.

41. The method of claim 325, wherein the circulating step provides sufficient freezing in order to create a first full-thickness lesion having a thickness extending through the entire thickness of a heart wall for the entire length of the distal treatment section of the catheter in contact with the heart wall.

42. A system for creating at least one lesion in target tissue, the system comprising:
a cryoablation catheter comprising:
an elongate shaft having an intermediate section and a distal treatment section comprising:
a low-profile, undeployed configuration; and
a high-profile deployed configuration, wherein the deployed configuration has an eccentric shape comprising a major axis and a minor axis less than the major axis, and wherein the distal treatment section in the deployed configuration comprises a preferential bias such that the major axis is reduced prior to the minor axis when the distal treatment section is subjected to forces arising from contacting the tissue; and
at least one energy delivery member extending along the elongate shaft; and
a console for controlling a flow of cryogen to the at least one energy delivery member to transfer heat from the target tissue to the distal treatment section thereby creating the at least one lesion in the target tissue.

43. The system of claim 42, wherein the cryogen is near critical nitrogen.

44. The system of claim 41, wherein the eccentric shape of the catheter distal treatment section in the deployed configuration has an effective elasticity less than that of heart wall tissue.

45. The system of claim 41, wherein the eccentric shape of the catheter distal treatment section in the deployed configuration has an effective elasticity that is substantially the same as that of a wall of a left atrium or a human heart.

46. The system of claim 41, further comprising an elongate control member extending along the intermediate section, and secured to a distal tip of the distal treatment section, the elongate control member being in movable cooperation with the intermediate section for causing movement of the distal tip relative to the intermediate section to adjust the shape of the distal treatment section in the deployed configuration.

47. The system of claim 41, wherein the eccentric shape defines a plane that is substantially perpendicular to the elongate shaft.

48. The system of claim 46, wherein the control member is rotatable to modify the length of the major axis independent from modifying the length of the minor axis.

49. A method of treating atrial fibrillation comprising the step of creating at least one lesion as recited herein.

50. A catheter for treating atrial fibrillation including any structure and function as recited herein.

51. A system for treating atrial fibrillation comprising a catheter as described herein, and a controller configured to adjust the amount of energy delivered from the tissue to the catheter.
